(19) United States
(12) Patent Application Publication
(10) Pub. No.: US 2023/0009161 A1
(43) Pub. Date: Jan. 12, 2023

(54) GENE TRANSFER SYSTEM

(71) Applicant: The University of North Carolina at Chapel Hill, Chapel Hill, NC (US)

(72) Inventors: Samuel Lai, Chapel Hill, NC (US); Christina PARKER, Chapel Hill, NC (US); Justin HUCKABY, Chapel Hill, NC (US); Timothy JACOBS, Chapel Hill, NC (US)

(21) Appl. No.: 17/783,038

(22) PCT Filed: Dec. 10, 2020
(86) PCT No.: PCT/US2020/064340

§ 371 Date: Jun. 7, 2022

Related U.S. Application Data
(60) Provisional application No. 62/946,202, filed on Dec. 10, 2019.

(51) Int. Cl. C12N 15/86; C07K 14/18; C07K 16/10

(52) U.S. Cl. CPC C12N 15/86; C07K 14/1808; C07K 16/1045; C07K 2317/31; C12N 2740/16043; C12N 2770/36122

(57) ABSTRACT

The present disclosure relates to a viral gene delivery vector particle and a bispecific polypeptide configured to bind a viral gene delivery vector particle and target cell-specific receptor protein. The disclosure also relates to gene delivery systems, compositions, and methods of use thereof.

### CROSS REFERENCE TO RELATED APPLICATIONS

This application claims the benefit of U.S. Provisional Application No. 62/946,202, filed on Dec. 10, 2019, the contents of which is incorporated herein by reference.

### FIELD

The present disclosure relates to a gene transfer system comprising a viral gene delivery vector particle and a bispecific polypeptide configured to bind a viral gene delivery vector particle and target cell-specific receptor protein.

### BACKGROUND

Selective transduction of only target cells and tissues represents a major goal of therapeutic gene delivery. To do so, gene vectors must avoid binding to off-target cells while quickly binding to target cells with high specificity, and efficiently deliver DNA to the nucleus following cell entry. Among common viral vectors, lentivirus (LV) is one of the most efficient gene transduction system for stable, long-term transgene expression. Importantly, the safety of LV has greatly improved since adverse effects were first observed in patients with X-clinical severe combined immunodeficiency (SCID) who underwent retrovirus-mediated gene therapy. As a result, LV vectors are now routinely used in CAR-T cell therapies (i.e. T-cells modified to possess a chimeric antigen receptor) for B-cell malignancies where cells are selected, transduced with LV vectors, expanded, and reinfused into patients; two such therapies have already received regulatory approval.

Despite the routine in vivo delivery of cells transduced with LV vectors ex vivo, LV vectors are rarely used directly for in vivo gene therapy. This is because common LV vectors lack cell specificity: wildtype LV envelope proteins generally bind proteins ubiquitously present on the surface of most cells, leading to extensive off-target effects. Strategies to alter or restrict the natural tropism of LV vectors include either pseudotyping LV with different viral envelope proteins possessing altered tropism and biodistribution, or genetically inserting ligands, peptides, and single-chain antibodies into viral envelope glycoprotein domains to confer new cellular specificity. Unfortunately, introducing large proteins can be deleterious to the structure of viral proteins, impede proper folding of the incorporated peptide that diminishes cell binding, and may hinder viral infectivity by altering normal functions of viral attachment proteins or preventing conformational changes necessary for fusion. Indeed, modified vectors can suffer from inconsistent specificity, reduced fusion activity, and low viral titers. Not surprisingly, the success of modifying viral envelope glycoproteins domains depends on the size, structure, and binding activity of ligand.

### SUMMARY

Disclosed herein is a gene delivery system comprising a viral gene delivery vector particle comprising a polynucleotide encoding at least one gene-of-interest and a bispecific polypeptide configured to bind a viral gene delivery vector particle and target cell-specific receptor protein, wherein the viral gene delivery vector particle is a lentivirus. In some embodiments, the lentivirus comprises a modified Sindbis virus envelope protein unable to bind a cell surface protein.

Also disclosed herein is a composition comprising a viral gene delivery vector particle comprising a polynucleotide encoding at least one gene-of-interest and a bispecific polypeptide configured to bind a viral gene delivery vector particle and target cell-specific receptor protein, wherein the viral gene delivery vector particle is a lentivirus. In some embodiments, the lentivirus comprises a modified Sindbis virus envelope protein unable to bind a cell surface protein.

Further disclosed are methods of transducing a cell with at least one gene-of-interest, methods of targeting at least one gene-of-interest to a cell or tissue, methods of generating CAR cells (CAR-T cells), and methods of treating a disease or disorder using the compositions or gene delivery systems.

Other aspects and embodiments of the disclosure will be apparent in light of the following detailed description and accompanying figures.

### BRIEF DESCRIPTION OF THE DRAWINGS

FIGS. 1A-IE show the characterization of control and bispecific antibodies (bsAb). FIG. 1A is a schematic representation of Sindbis glycoprotein domains E1 and E2. Mutated Sindbis envelope glycoprotein (mSindbis) contains mutations in the E2 domain (indicated by arrows) that ablate native receptor binding. E1 domain forms a heterodimer with E2, and E3 is a signal sequence peptide for E2 protein. FIG. 1B is a schematic of control and bispecific Ab illustrating size and key design features. FIG. 1C is a nonreducing (left) and reducing (right) protein gel showing Coomassie blue staining of control and bispecific Ab. FIG. 1D is a graph of the binding affinity of control and bispecific Ab to HER2-Fc chimera analyzed by ELISA (n=2). FIG. 1E shows the selective binding of αE2 and αE1 bispecific Ab to Sindbis pseudotyped lentiviruses and no binding to negative control (no envelope lentivirus) as visualized by dot blot.

FIGS. 2A-2D show BsIgG₁^{E2×HER2} enhanced transduction by WT Sindbis and mSindbis pseudotyped lentiviral vectors against HER2⁺ SKBR3 cells compared to either virus alone. bsAb-mediated viral infectivity was measured by flow cytometry as percentage of GFP positive cells (FIG. 2A) and mean fluorescence intensity, MFI (FIG. 2B). Data represents n=5 independent experiments performed in duplicates, MOI=3, and antibody concentration=1 nM (two-way ANOVA post-hoc Tukey's test, **** indicates p<0.0001 vs all conditions). Targeted lentiviral infectivity is dependent upon HER2 specificity of bsAb (FIG. 2C-2D). At all tested concentrations of bsAb, excess Trastuzumab (αHER2 IgG₁) effectively blocked viral infectivity of both targeted lentiviruses, suggesting that the infectivity was mediated specifically via binding to HER2 receptor and not due to differences between lentiviruses. Data represents n=3 independent experiments performed in duplicates, MOI=3, and analyzed using two-way ANOVA with post-hoc Tukey's test (#p<0.0001 vs all conditions, ****p<0.0001, **p=0.0013).

FIGS. 3A-3C show the specific infection of HER2⁺ cells in a mixed cell population. Targeted WT and mSindbis substantially enhanced viral infectivity in HER2⁺ cells compared to control HER2⁻ cells (FIGS. 3A-3B). Solid lines compare the selectivity of redirected LV in HER2⁺ cells vs HER2⁺ cells, and dashed lines compare the transduction efficiency of redirected LV using bsIgG₁^{E2×HER2} versus LV alone in target HER2⁺ cells. Viral infectivity was measured by flow cytometry as percentage of GFP positive cells (FIG. 3A) and mean fluorescence intensity, MFI (FIG. 3B). A2780 (HER2⁻) cells were mixed with SKBR3 (HER2⁺) to create a mixed cell population (FIG. 3C). Both targeted lentiviruses demonstrated selectivity for HER2⁺ cells (FIG. 3D) compared to HER2⁻ cells (FIG. 3E) as indicated by the substantial increase in percentage of GFP positive cells. Data represents 2 independent experiment performed in duplicates, MOI 3, [Ab]=1 nM, and analyzed using two-way ANOVA with post-hoc Tukey's test (****p<0.0001 vs all conditions, **p=0.0012).

FIGS. 4A-4E show the characterization of bispecific tandem Fab. FIG. 4A is a schematic representation of Sindbis glycoprotein domains E1 and E2. Mutated Sindbis envelope glycoprotein (mSindbis) contains mutations in the E2 domain (indicated by arrows) that ablate native receptor binding. E1 domain forms a heterodimer with E2, and E3 is a signal sequence peptide for E2 protein. FIG. 4B is a schematic of control and bispecific Ab illustrating size and key design features between bsIgG₁ and tandem Fab. FIG. 4C is a nonreducing (left) and a reducing (right) protein gel showing Coomassie blue staining of control and bispecific Ab. Binding affinity of control and bispecific Ab to HER2-Fc chimera analyzed by ELISA (FIG. 4D). FIG. 4E shows selective binding of bispecific Ab (bsIgG₁ and tandem Fab) to Sindbis pseudotyped lentiviruses and no binding to negative control (no envelope lentivirus) as visualized by dot blot.

FIGS. 5A-5C show the comparable transduction efficiency of target viruses coated with bsIgG₁^{E2×HER2} and tandem Fab^{E2×HER2} in target HER2⁺ cells. Viral infectivity was measured by flow cytometry as a percentage of GFP positive cells (FIG. 5A) and mean fluorescence intensity, MFI (FIG. 5B). Targeted lentiviral infectivity is dependent upon HER2 specificity of bispecific antibody (FIG. 5C). Excess Trastuzumab (IgG₁^{HER2}) substantially reduced viral infectivity of both targeted lentiviruses. All data represents n=2 independent experiments, MOI=3, [bsIgG₁^{E2×HER2} ]=1 nM, [tandem Fab^{E2×HER2} ]=5 nM, and [IgG₁^{HER2}]=nM, and analyzed using two-way ANOVA with post-hoc Tukey's test (#p<0.0001 vs all conditions, ****p<0.0001, ***0.0002<p<0.001, ***p=0.0003).

FIG. 6 shows lentiviral redirection with bispecific antibodies exhibited minimal to no effect on cell viability compared to untreated cells. Immediately following viral infectivity assay with bispecific antibodies in SKBR3 cells, the cell viability of untreated and transduced cells was measured using MTT assay. Cells were incubated with 0.5 mg/ml MTT solution for 1 h at 37° C. prior to the addition of isopropanol to dissolve formazan crystals, and absorbance was measured at 560 nm (signal) and 670 nm (background). Cell viability was reported as percent viability of treated cells relative to untreated cells. Data represents n=2 independent experiments performed in triplicates, MOI=3, and antibody concentration=5 nM (two-way ANOVA post hoc Tukey's test, * indicates 0.02<p<0.04, **p=0.0019).

FIG. 7 shows a schematic comparison of different strategies to generate autologous CAR-T cells. Traditional CAR-T cell development (left) involves a time-consuming biomanufacturing process that begins with blood collection from the patient. Following isolation, activation, and transduction of T cells with viral vectors, CAR-T cells are expanded for several weeks ex vivo prior to cryopreservation. After extensive quality controls, CAR-T cells are shipped to the clinic for reinfusion into the patient. Targeted lentiviral vector gene delivery system as described herein (right) offers a much faster and simplified approach for generating CAR-T cells directly in vivo following a single infusion of engineered viral vector system. The system comprises a mutant lentivirus expressing an envelope glycoprotein with mutations that abrogate native receptor tropism, and a bispecific binder (tFab) that redirects the lentivirus to T cells. The system involves simply mixing the lentivirus and tFab shortly prior to infusion.

FIGS. 8A-8F show that bispecific antibody binder enhanced specificity and transduction efficiency of the mutant lentivirus. FIG. 8A is a schematic of bispecific antibody in tandem Fab format (tFab) used for redirecting mutant Sindbis lentiviral vector (SINV-LV) to CD3⁺ T cells for targeted transduction. Orthogonal amino acid mutation sets are shown for constant and variable domains of each Fab to ensure correct pairing of heavy and light chains. FIG. 8B shows binding affinity of control IgG (circle) and tFab (square) to human CD3ε analyzed by ELISA (n=2). FIG. 8C shows binding affinity of tFab (square) to mutant Sindbis E2 glycoprotein analyzed by ELISA (n=2). SINV-GFP transduction to CD3⁺ T cells was enhanced by addition of the tFab molecule in a concentration-dependent manner (FIG. 8D). At all tested concentrations, excess anti-CD3 IgG of the same clone blocked tFab-mediated SINV-GFP transduction, suggesting that transduction was specifically mediated via tFab binding CD3 in T cells. Data represent results of 3 independent experiments performed in triplicate (MOI=25) and analyzed using a two-way ANOVA with a post-hoc Tukey's test for multiple comparisons (****, p<0.0001). FIG. 8E shows that addition of tFab to SINV-GFP redirected the mutant lentiviral vector to CD3⁺ T cells in a mixed culture (CD3⁺ and CD3⁻ cells together) demonstrating the specificity towards CD3⁺ T cells. FIG. 8F is a graph showing that in mixed cultures of CD3 (Sup-T1) and CD3⁻ (BV-173) cells, SINV-GFP plus tFab demonstrated substantial selectivity towards CD3⁺ T cells as indicated by the increase in percentage of GFP⁺ cells. Data represent results of 3 independent experiments performed in triplicate (MOI=25; [tFab]=30 nM) and analyzed using a two-way ANOVA with a post-hoc Tukey's test for multiple comparisons (****, p<0.0001).

FIGS. 9A-9D show that T cells transduced with SINV-CAR in combination with tFab expressed functional CD19.CAR and eliminate tumor B cells in vitro. FIG. 9A is a schematic representation of the CD19.CAR cassette under the control of the EF-1α promoter and WPRE post-transcriptional regulatory molecule. FIG. 9B is an experimental schema for the transduction and subsequent co-culturing of CAR-T cells with tumor B cells in vitro. FIG. 9C is representative flow plots (left panel) and summary (right panel) of the quantification of residual CD19+ tumor B cells (BV-173 and Daudi cell lines) remaining after co-culturing with either NT, tFab, SINV-CAR, or SINV-CAR plus tFab treated T cells (E:T=2:1). All cells were collected after 4 or 5 days (BV-173 and Daudi, respectively) and stained with CD3 and CD19 mAbs to identify T cells and tumor cells, respectively, by flow cytometry (n=4, mean shown). ***P=0.0004, ****P<0.0001, two-way ANOVA. FIG. 9D are graphs showing quantification of IFNγ (left panel) and IL-2 (right panel) cytokines in supernatant collected after 24 hours of co-culturing NT, tFab, SINV-CAR, or SINV-CAR plus tFab treated T cells with tumor cell lines (E:T=2:1) (n=4, mean shown). *P=0.0393, **P=0.0015, ****P<0.0001, two-way ANOVA.

FIGS. 10A-10E show that SINV-CAR targeted with tFab generated functional CAR-T cells directly in vivo. FIG. 10A is an experimental schema of the mouse model. Following a dose of irradiation (100 rad), mice were injected with FFLuc BV-173 (5×10⁵ cells) intravenously (i.v.). Five days later mice were injected i.v. with 5×10⁶ activated PBMCs followed by either SINV-CAR alone or SINV-CAR plus tFab 30 minutes later. FIG. 10B is representative tumor bioluminescence (BLI) (color scale: min=1×10⁶; max=5×10⁷) for mice treated according to scheme from FIG. 10A. FIG. 10C is a graph of BLI kinetics for all mice treated according to scheme from FIG. 10A. Lighter lines represent individual mice, while bolded lines represent the means for the treatment groups. Summary of 2 independent experiments (n=10 mice for each condition). ***P=0.0002; ****P<0.0001, two-way ANOVA with Bonferroni correction. FIG. D is a Kaplan-Meier survival curve for all mice (n=10 mice per condition) treated according to scheme from FIG. 10A. *P=0.0242, log-rank test. FIG. 10E is representative flow plots (left panel) and quantification (right panel) of CAR-T cells (gated on CD3⁺CD45⁺) in the peripheral blood at the time of euthanasia (n=10 each condition, mean shown). Empty symbols denote the flow plots shown to the left. *P=0.0214, unpaired t test.

FIGS. 11A-11C show that SINV-CAR targeted with tFab suppressed tumor growth in spleen. Mice engrafted with FFLuc BV-173 tumor cells and treated with either SINV-CAR alone or SINV-CAR plus tFab were euthanized, and spleens were weighed (FIG. 11A-right, n=10, mean shown). Representative images of the spleens (FIG. 11A-left panel). ***P=0.0002, unpaired t test. FIG. 11B is representative flow plots (left panel) and quantification (right panel) of human CAR-T cells (gated on CD3+CD45⁺) in the spleen at the time of euthanasia (n=10 each condition, mean shown). Empty symbols denote the flow plots shown to the left. **P=0.0076, unpaired t test. FIG. 11C is representative flow plots (left panel) and summary (right panel) of the percentage of human CD19⁺ tumor B cells infiltrating the spleen of mice engrafted with FFLuc BV-173 and treated with either SINV-CAR alone or SINV-CAR plus tFab at time of sacrifice (n=10, mean shown). ****P<0.0001, unpaired t test.

FIGS. 12A-12E show characterization of mutant Sindbis lentivirus (SINV-LV) and bispecific antibody binder (tFab). FIG. 12A is a schematic representation of mutant Sindbis (SINV) envelope glycoproteins (E1 and E2) with arrows to denote mutations that ablate native receptor binding capabilities of the E2 domain. The E1 domain is responsible for pH-dependent membrane fusion. E1 and E2 heterodimerize together to form trimeric spikes on the viral surface. FIG. 12B is schematic representation of control anti-CD3 IgG (left panel) and anti-CD3× anti-E2 bispecific antibody (tFab) (right panel). FIG. 12 C is a non-reduced (left panel) and reduced (right panel) SDS-PAGE with Coomassie blue protein staining showing molecular weight and purity of control IgG and bispecific tFab. FIG. 12D shows tFab bound specifically to SINV enveloped lentivirus and did not bind non-specifically to other common lentiviral pseudotypes (VSV-G and Measles Virus) as demonstrated by immunodot blotting. α-CD3 IgG negative control displays no binding to any of the three lentiviral pseudotypes tested. FIG. 12E is transmission electron microscopy (TEM) images of SINV-LV without addition of tFab (left panel) and with addition of tFab (right panel) to confirm binding and presence of tFab on targeted lentiviral surface (arrows).

FIGS. 13A-13E show that T cells transduced with SINV-CAR in combination with tFab expressed functional CD19.CAR and eliminated tumor B cells in vitro. FIG. 13A is a representative flow plot showing the composition of B cells and T cells in human PBMCs 2 days after isolation. FIG. 13B is a graph of CD3 expression in PBMCs detected with a commercial antibody after 24 hours of rest in complete medium following prior activation with either soluble or plate-bound anti-CD3 and anti-CD28 antibodies. FIG. 13C is flow cytometry plots (left) and summary (right) showing CAR expression in T cells transduced with SINV-CAR or SINV-CAR plus tFab. Non-transduced (NT) and tFab alone samples of T cells are shown as negative controls (n=4, mean shown). *, P=0.0437 SINV-CAR plus tFab vs SINV-CAR; *, P=0.0100 SINV-CAR plus tFab vs tFab with paired t test. FIG. 13D is representative flow plots (left panel) and quantification summary (right panel) of residual tumor cells remaining in cocultures with NT, tFab, SINV-CAR, or SINV-CAR plus tFab treated T cells (E:T=1:1) for 4 or 5 days (BV-173 and Daudi, respectively). All cells were collected and stained with CD3 and CD19 mAbs to identify T cells and tumor cells, respectively, by flow cytometry (n=4, mean shown). *, P=0.0350 SINV-CAR plus tFab vs SINV-CAR; *, P=0.0175 SINV-CAR plus tFab vs tFab; **P=0.0003, two-way ANOVA. FIG. 13E are graphs of the quantification of IFNγ (left panel) and IL-2 (right panel) cytokine production in supernatant collected after 48 hours of co-culturing NT, tFab, SINV-CAR, or SINV-CAR plus tFab treated T cells with tumor cell lines (E:T=1:1) (n=4, mean shown). **P=0.0013, ****P<0.0001, two-way ANOVA.

FIGS. 14A-14D show absolute numbers of T cells detected from weekly bleeds and at sacrifice for mice of in vivo tumor model. FIG. 14A is representative flow plots (left panel) of the percentage of CD3⁺ CD45⁺ human T cells in the peripheral blood at day 18. Quantification summary (right panel) of the number of CD3⁺ CD45⁺ human T cells in the peripheral blood 4, 11, and 18 days after PBMCs injection (n=10 each condition, mean shown). Empty symbols denote the flow plots shown to the left. FIG. 14B is a graph of the quantification summary of the number of hCD3⁺hCD45⁺ T cells in the peripheral blood at the time of euthanasia (n=10 each condition, mean shown). FIG. 14C is representative flow plots (left panel) of the percentage of human CAR-T cells (gated on CD3⁺ CD45⁺) in the peripheral blood at day 18. Quantification summary (right panel) of the number of human CAR-T cells (gated on CD3⁺CD45⁺) in the peripheral blood 4, 11 and 18 days after PBMCs injection (n=10 each condition, mean shown). Empty symbols denote the flow plots shown to the left. FIG. 14D is a graph of the quantification summary of the number of hCD3⁺hCD45⁺ T cells in the spleen at the time of euthanasia (n=10 each condition, mean shown).

### DETAILED DESCRIPTION

The goal of gene therapy is specific delivery and expression of therapeutic genes to target cells and tissues. Common lentiviral (LV) vectors are efficient gene delivery vehicles but offer little specificity. To enable highly specific transduction, cell-specific receptor binding must be robust while minimizing off-target binding. With wildtype viral vectors that are either pseudotyped with Ab or mixed with adaptor molecules, the resulting vectors can still bind and transduce off-target cells/tissues via the native viral envelope proteins.

Described herein is a versatile redirection platform combining modified Sindbis (mSindbis)-pseudotyped LV with bispecific antibodies (bsAb) that bind both mSindbis E2 and specific cell receptors. A E2- and HER2-targeted bsAb provided the specificity required to redirect mSindbis LV to transduce HER2⁺ cells, thus enabling the use of LV with an unmodified viral envelope that likely maximizes stability, high titer production, and efficient transduction. A longstanding challenge in bsAb engineering has been the proper pairing of heavy and light chains leading to high purity and yield of the final product. As an example method to generate bispecific antibodies, orthogonal mutation pairs were introduced into heavy and light chains to yielded high fidelity pairing of the correct heavy and light chains for functional bsAb (See Lewis et al., Nat Biotechnol 2014 February; 32(2):191-8, incorporated herein by reference in its entirety). Here, the versatile gene carrier system, combining bsAb with a mutated LV that abrogates its native receptor binding tropism, facilitated highly potent and specific gene delivery.

A single dose of a targeted lentiviral vector administered in vivo, as described herein, generated CAR-T cells from circulating T lymphocytes in a humanized tumor mouse model of B cell leukemia. The in vivo engineered CAR-T cells greatly suppressed CD19⁺ tumor cell growth and prolonged the overall survival time of mice, despite the highly aggressive nature of the tumor model.

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

A “bispecific polypeptide,” as used herein, refers to a polypeptide having binding specificities for at least two different moieties or targets.

The term “viral vector particle” as used herein refers to a recombinant virus which carries a polynucleotide encoding at least one gene-of-interest, which is generally flanked by viral LTRs.

The term “transducing” denotes the delivery of a polynucleotide to a recipient cell either in vivo or in vitro, via a replication-defective viral vector, for example, a viral gene delivery vector particle.

The term “chimeric antigen receptor” and “CAR” are used interchangeably herein to refer to molecules that combine antibody-based specificity for a desired antigen (e.g., tumor antigen) with a cell receptor (e.g. T cell receptor)-activating intracellular domain to generate a chimeric protein that exhibits a specific anti-tumor cellular immune activity.

“Polynucleotide” or “oligonucleotide” or “nucleic acid,” as used herein, means at least two nucleotides covalently linked together. The polynucleotide may be DNA, both genomic and cDNA, RNA, mRNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The proteins may be modified by the addition of sugars, lipids or other moieties not included in the amino acid chain. The terms “polypeptide”, and “protein,” are used interchangeably herein.

As used herein, the terms “providing”, “administering,” “introducing,” are used interchangeably herein and refer to the placement of the compounds and/or compositions of the present disclosure into a subject by a method or route which results in at least partial localization of the compound and/or composition to a desired site. The compound and/or compositions can be administered by any appropriate route which results in delivery to a desired location in the subject.

A “subject” or “patient” may be human or non-human and may include, for example, animal strains or species used as “model systems” for research purposes. Likewise, patient may include either adults or juveniles (e.g., children). Moreover, patient may mean any living organism, preferably a mammal (e.g., human or non-human) that may benefit from the administration of compounds and/or compositions contemplated herein. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one aspect of the methods provided herein, the mammal is a human.

As used herein, “treat,” “treating” and the like means a slowing, stopping or reversing of progression of a disease or disorder when provided a composition described herein to an appropriate control subject. The term also means a reversing of the progression of such a disease or disorder to a point of eliminating or greatly reducing the cell proliferation. As such, “treating” means an application or administration of the compositions described herein to a subject, where the subject has a disease or a symptom of a disease, where the purpose is to cure, heal, alleviate, relieve, alter, remedy, ameliorate, improve or affect the disease or symptoms of the disease. “

A “lentivirus” refers to a retroviral genus capable of infecting dividing and non-dividing cells. Examples of lentiviruses include HIV (human immunodeficiency virus: HIV type 1 and HIV type 2), etiologic agent of human acquired immunodeficiency syndrome (AIDS); Visna-maedi, a causative agent of encephalitis (bizna) or pneumonia (medi), caprine arthritis-causing encephalitis, encephalitis virus); Equine infectious anemia virus which causes autoimmune hemolytic anemia and brain disease in horses; Feline immunodeficiency virus (FIV), which causes immune system deficiency in cats; Bovine immune deficiency virus (BIV), which causes lymphadenopathy, lymphocytosis and possible central nervous system infections in cattle; And simian immunodeficiency virus (SIV), an ape-like virus that causes immune system deficiency and brain disease in subhuman primates. As used herein, the term “lentivirus” also includes lentiviruses that are pseudotyped with a glycoprotein derived from another virus, such as lentiviruses pseudotyped with measles, lentiviruses pseudotyped with nipah viruses, etc.

The lentiviral genome is generally composed of 5′long terminal repeat (LTR), gag gene, pol gene, env gene, additional genes (nef, vif, vpr, vpu) and 3′ LTR. The virus LTR is divided into three regions called U3, R and U5. The U3 region includes an enhancer and a promoter element. The U5 region contains a polyadenylation signal. The R (repeat) region separates the U3 and U5 regions, and the sequence of the transcribed R region appears at both the 5′ and 3′ ends of the viral RNA. See, for example, “RNA Viruses: A Practical Approach” (Alan J. Cann, Ed., Oxford University Press, (2000)), O Narayan and Clements J. Gen. Virology 70: 1617-1639 (1989), Fields et al. Fundamental Virology Raven Press. (1990), Miyoshi H, Blomeru, Takahashi M, Gage F H, Verma I M. J Virol. 72 (10): 8150-7 (1998), and U.S. Pat. No. 6,013,516.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. GENE DELIVERY SYSTEM AND COMPOSITIONS

The present disclosure provides gene delivery systems comprising a viral gene delivery vector particle comprising a polynucleotide encoding at least one gene-of-interest and a bispecific polypeptide configured to bind a viral gene delivery vector particle and a target cell-specific receptor protein.

Viral particles, also known as virions, consist of a nucleic acid(s) surrounded by a capsid coat. The viral particles can be enveloped or nonenveloped depending on the presence or absence of an envelope comprised of host cell membranes, as well as viral glycoproteins. The viral particle may be a member of the Retroviridae (retrovirus) family, or a derivative thereof. The viral particle may be a pseudotyped viral particle or a pseudovirus comprising a heterologous envelope protein or an envelope protein originating for a different virus. In some embodiments, the viral particle is a lentivirus. In some embodiments, the viral gene delivery vector particle is a lentivirus. Lentiviruses are a subtype of retroviruses that are capable of infecting non-dividing and actively dividing cell types. In select embodiments, the viral gene delivery vector particle is a lentivirus comprising a modified Sindbis virus envelope protein unable to bind a cell surface protein.

In some embodiments, the viral particle comprises at least one protein (such as an envelope protein (e.g., gp160 protein, gp4l protein, etc.), capsid protein, matrix protein, etc.) or glycoprotein that has been modified in such that the virus does not bind to its target cell. For example, modifications can be made to block the interactions between a viral envelope glycoprotein and a specific target cell surface receptor which determines the cellular target for the virus. For example, modifications can include in Sindbis, one or more mutations and/or deletions in (a) the E3 leader sequence (e.g., amino acid residues 61-64 can be deleted (deletion of these amino acid residues is known to reduce tropism and result in higher titer production)), (b) the E2 glycoprotein where, mutations such as SLKQ68-71AAAA or KE159-160AA (which are known to reduce natural tropism while retaining higher titer production) can be made (See, Morizono et al., Nat Med. 2005 March; 11(3):346-52. Epub 2005 Feb. 13), and (c) the E1 glycoprotein, where mutations such as AK226-227SG (which are believed to allow E1 to mediate fusion in absence of cholesterol in target membrane”, see for example, Pariente et al Mol Ther. 2007 November; 15(11):1973-81. Epub 2007 Jul. 24.) can be made. In lentiviruses pseudotyped with measles, mutations can be made in the H (hemagglutinin) protein, such as at amino acids Y481A, R533A, SF548-549LS (which abrogate native receptor binding to SLAM and CD46 typically found on immune cells; see Vongpunsawad S, et al., J Virol. 2004 January; 78(1):302-13 and Nakamura T, et al., Nat Biotechnol. 2005 February; 23(2):209-14. In lentiviruses pseudotyped with nipah, mutations can be made in glycoprotein G, such as, at amino acids, E501A, W504A, Q530A, and E533A (see Bender et al. PLOS Pathogens. 2016 June journals.plos.org/plospathogens/article?id=10.1371/journal.ppat.1005641).

The bispecific polypeptide is any polypeptide capable of interacting with two different binding partners at the same time. In some embodiments, the bispecific polypeptide comprises at least one binding domain configured to bind the viral gene delivery vector particle and at least one binding domain configured to bind the target cell-specific receptor protein. In some embodiments, the bispecific polypeptide binds an envelope protein of the virus particle (e.g. the modified Sindbis virus envelope protein). In select embodiments, the bispecific polypeptide binds the modified Sindbis virus envelope protein in the E2 domain.

In some embodiments, the bispecific polypeptide further comprises a flexible linker covalently joining the two binding domains. The linkers may be flexible such that they do not constrain either of the two components they link together in any particular orientation. The linkers may comprise any amino acid sequence. The linkers may essentially act as a spacer. In some embodiments, the linkers are glycine-rich and/or serine-rich (e.g. (G₄S)₆). In some embodiments, the bispecific polypeptide can comprise one flexible linker, two flexible linkers, three flexible linkers, four flexible linkers, five flexible linkers, six flexible linkers, seven flexible linkers, eight flexible linkers nine flexible linkers, ten flexible linkers, eleven flexible linkers, twelve flexible linkers, etc. When multiple flexible linkers are used the flexible linkers may be the same, or the flexible linkers can be different.

The bispecific polypeptide may be an antibody, fragment, or derivative thereof. In some embodiments, the antibody, fragment or derivative thereof is two or more Fab-fragments, two or more F(ab₂)′-fragments, single domain antibodies, an IgG with Fc, a chimeric antibody, a CDR-grafted antibody, a bivalent antibody-construct, a humanized antibody, a human synthetic antibody, or a chemically modified derivative thereof, a multispecific antibody, a diabody (e.g., two or more scFv fragments covalently linked together), tandem scFv fragments, bivalent (or bispecific) (scFv)₂, so-called miniantibody, VHH nanobodies, another type of a recombinant antibody, or the like as known in the art (See Spiess et al., Mol Immunol 2015 October; 67(2):95-106, incorporated herein by reference in its entirety). By the term “recombinant antibody” as used herein, is meant an antibody or antibody fragment which is generated using recombinant DNA technology, such as, for example, an antibody or antibody fragment expressed by a bacterial system, a yeast expression system, a fungus-based expression system, a plant-based expression system, or a mammalian cell expression system. The term should also be construed to mean an antibody or antibody fragment which has been generated by the synthesis of a DNA molecule encoding the antibody or antibody fragment and which DNA molecule expresses an antibody or antibody fragment protein, or an amino acid sequence specifying the antibody or antibody fragment, wherein the DNA or amino acid sequence has been obtained using recombinant or synthetic DNA or amino acid sequence technology which is available and well known in the art. In select embodiments, the bispecific polypeptide comprises two or more Fab domains individually configured to bind the viral gene delivery vector particle and the target cell-specific receptor protein. In other select embodiments, the bispecific polypeptide comprises at least three or more Fab domains individually configured to bind the viral gene delivery vector particle and the target cell-specific receptor protein. In other select embodiments, the bispecific polypeptide comprises at least four or more Fab domains individually configured to bind the viral gene delivery vector particle and the target cell-specific receptor protein. In other select embodiments, the bispecific polypeptide comprises at least five or more Fab domains individually configured to bind the viral gene delivery vector particle and the target cell-specific receptor protein.

In some embodiments, the antibody is a human or humanized antibody. The term “humanized antibody”, as used herein, is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the presently disclosed subject matter may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

The target cell-specific receptor protein may be any protein known in the art to be associated with a particular subset of cells. The cell-specific receptor protein may be associated with cells from a certain tissue or cells from a certain state of disease, including but not limited to, CD3, CD4, CD8 for T-cells, CD19 for B-cells, cancer cell markers (e.g., HER2), or the like. In some embodiments, the target cell-specific receptor protein is selected from the group consisting of a T cell receptor, a B cell receptor, and a cancer cell marker. The target cell-specific receptor protein may be exogenous or endogenous to the cell type. For example, recombinant cells may comprise an exogenous receptor protein targeted by the bispecific polypeptide.

The gene-of-interest may comprise one or more fully functioning genes. The gene may comprise any gene encoding a functioning protein, a fragment, or derivative thereof. The gene may comprise a marker protein, a therapeutic protein, elements required for genomic editing or gene silencing. In some embodiments, the gene-of-interest comprises a chimeric antigen receptor. The gene-of-interest may comprise genetic elements that aid in targeted integration of therapeutic transgenes of interest or targeted knockout of genes-of-interest (e.g. components of CRISPR/Cas9).

The present disclosure also provides a composition (e.g. a pharmaceutical composition) comprising a viral gene delivery vector particle comprising a polynucleotide encoding at least one gene-of-interest and a bispecific polypeptide configured to bind a viral gene delivery vector particle and target cell-specific receptor protein. Descriptions provided above for the viral gene delivery vector particle, the polynucleotide, the at least one gene-of-interest, and the bispecific polypeptide provided above are maintained for the composition.

The compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator. The route by which the disclosed compositions are administered and the form of the composition will dictate the type of carrier to be used.

The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis). Techniques and formulations may generally be found in “Remington's Pharmaceutical Sciences,” (Meade Publishing Co., Easton, Pa.). Therapeutic compositions must typically be sterile and stable under the conditions of manufacture and storage.

### 3. METHODS OF USE

The present disclosure provides methods of transducing a cell with at least one gene-of-interest, comprising contacting a cell expressing the target cell-specific receptor protein with the gene delivery system or the composition described in section 2. The present systems or compositions may be delivered to a cell with any suitable means. In certain embodiments, the system is delivered in vivo. In other embodiments, the system is delivered to isolated/cultured cells in vitro to provide modified cells useful for in vivo delivery to patients afflicted with a disease or condition.

The present disclosure also provides methods of targeting at least one gene-of-interest to a cell or tissue, comprising administering to a subject having a cell or tissue expressing the target cell-specific receptor protein the gene delivery system or the composition described in section 2.

The present disclosure further provides methods of generating CAR-T cells. In some embodiments, the methods generative CAR T cells in vivo and comprise administering to a subject the gene delivery system or the composition described in section 2, wherein the at least one gene of interest comprises a chimeric antigen receptor and the target cell-specific receptor protein is a T cell receptor. In some embodiments, the T cell receptor is selected from the group consisting of CD3, CD4, CD8, or a combination thereof.

The present disclosure further provides methods of treating a disease or disorder comprising administering to a subject an effective amount of the gene delivery system or the composition described in section 2, wherein the at least one gene of interest comprises a chimeric antigen receptor, a therapeutic protein, or a combination thereof.

Essentially any disease treatable with a therapeutic protein or genome editing may be used with the methods disclosed herein to target the protein to a cell or tissue as described herein. Furthermore, essentially any disease that involves the specific or enhanced expression of a particular antigen can be treated by targeting CAR cells to the antigen, as known in the art. For example, autoimmune diseases, infections, and cancers can be treated with methods, systems, and/or compositions of the invention. These include cancers, such as primary, metastatic, recurrent, sensitive-to-therapy, refractory-to-therapy cancers (e.g., chemo-refractory cancer). The cancer may be of the blood, lung, brain, colon, prostate, breast, liver, kidney, stomach, cervix, ovary, testes, pituitary gland, esophagus, spleen, skin, bone, and so forth (e.g., B-cell lymphomas or a melanomas) or any disease characterized as a cancer due to uncontrollable cell division. In the case of cancer treatment CAR cells typically target a cancer cell antigen (also known as a tumor-associated antigen (TAA)).

The specific dose level may depend upon a variety of factors including the age, body weight, and general health of the subject, time of administration, and route of administration. An “effective amount” is an amount that is delivered to a subject, either in a single dose or as part of a series, which achieves a medically desirable effect. For therapeutic purposes, and effect amount is the quantity which, when administered to a subject in need of treatment, improves the prognosis and/or state of the subject and/or that reduces or inhibits one or more symptoms to a level that is below that observed and accepted as clinically diagnostic or clinically characteristic of the disease or disorder. For prophylaxis purposes, an effective amount is that amount which induces a protective result without significant adverse side effects.

The frequency of dosing the effective amount can vary, but typically the effective amount is delivered daily, either as a single dose, multiple doses throughout the day, or depending on the dosage form, dosed continuously for part or all of the treatment period.

The composition or systems described herein may be formulated for any appropriate manner of administration, and thus administered, including for example, topical, oral, nasal, intravenous, intravaginal, epicutaneous, sublingual, intracranial, intradermal, intraperitoneal, subcutaneous, intramuscular administration, intratumoral, or via inhalation.

A wide range of second therapies may be used in conjunction with the systems and compositions of the present disclosure. The second therapy may be a therapeutic agent or may be a second therapy not connected to administration of an agent. Such second therapies include, but are not limited to, surgery, immunotherapy, radiotherapy, or a second chemotherapeutic agent.

### 4. KITS

In one aspect, the disclosure provides kits comprising at least one or all of the components of the disclosed system as described elsewhere herein (e.g. a viral gene delivery vector particle, a polynucleotide encoding at least one gene-of-interest, and/or a bispecific polypeptide). The components of the kit may be packaged separately or individually.

The disclosed kits can be employed in connection with disclosed methods of use.

The kits may further include information, instructions, or both for use of the kit in transducing a cell with at least one gene-of-interest, targeting at least one gene-of-interest to a cell or tissue, generative CAR cells, or treating a disease or disorder. The information and instructions may be in the form of words, pictures, or both, and the like.

### 5. EXAMPLES

### Materials and Methods

Cell lines 293T cells were cultured in DMEM containing 10% FBS. Human SKBR3 cells were purchased from the University of North Carolina at Chapel Hill (UNC-CH) Tissue Culture Facility, and A2780 cells were provided by Michael Jay (UNC-CH). SKBR3 cells were cultured in McCoy's medium containing 15% fetal bovine serum (FBS), and A2780 cells were cultured in RPMI 1640 containing 10% FBS and 1% L-glutamine. For co-culture studies, SKBR3 and A2780 cells were both cultured in McCoy's medium with 15% FBS. All cells were maintained at 37° C. and 5% CO₂.

B cell lymphoma tumor cell lines (BV-173 and Daudi) and T cell lymphoma tumor cells (Sup-T1) were purchased from ATCC and cultured in RPMI-1640 medium (Gibco) supplemented with 10% HyClone FBS (GE Healthcare), penicillin (100 U mL⁻¹; Gibco), and streptomycin (100 U mL⁻¹, Gibco). All cells were maintained at 37° C. and 5% CO₂ for growth. All cell lines are regularly tested for Mycoplasma, and the identity of each cell line was validated via flow cytometry for relevant surface markers and also monitored for morphological drift in culture. Cell lines were maintained in culture no longer than 30 days and then replaced with an earlier passage of cells thawed from cryopreservation. BV-173 cells were transduced with a gamma retroviral vector encoding the Firefly-Luciferase (FFluc) gene. Sup-T1 cells were engineered with a TCR construct to express full-length human CD3. Peripheral blood mononuclear cells (PBMCs) were isolated from fresh buffy coats (Gulf Coast Regional Blood Center) using Lymphoprep medium (Accurate Chemical and Scientific Corporation). PBMCs were then activated for 48 hours in bioreactors with soluble anti-CD3 (200 ng mL⁻¹; Miltenyi Biotec) and anti-CD28 (200 ng mL⁻¹; BD Biosciences) mAbs. Activated PBMCs were washed with PBS and allowed to rest at 37 C and 5% CO₂ in growth culture medium for at least 24 hours prior to lentiviral transduction or in vivo studies. Primary T cells were activated, cultured, and transduced in complete medium consisting of 45% Click's Medium (Irvine Scientific), 45% RPMI-1640 (Gibco), 10% HyClone FBS (GE Healthcare), 2 mmol L⁻¹ GlutaMax (Gibco), penicillin (100 U mL⁻¹; Gibco), and streptomycin (100 U mL⁻¹; Gibco) with 10 ng mL IL-7 and 5 ng mL⁻¹ IL-15 (PeproTech).

Preparation and characterization of fluorescent Sindbis pseudotyped lentivirus WT Sindbis and mSindbis pseudotyped lentiviruses (LV) were internally labeled with a GFP reporter gene. Particles were prepared by transfecting 293T cells with packaging plasmids pMDLg/pRRE and pRSV-Rev, transfer plasmid eGFP, and WT Sindbis or mSindbis envelope plasmid at a 1:1:1:1 ratio in serum-free media. The cell supernatant was collected 48 h later, and lentiviruses were purified from cell supernatant by ultracentrifugation using 25% (w/v) sucrose in HEPES-NaCl buffer. Lentiviruses were resuspended in 10% sucrose in HEPES-NaCl buffer, divided into aliquots, and stored at −80° C. Viral titer was quantified by qPCR-based lentivirus titration kit according to manufacturer's protocol (Applied Biological Materials, Inc., Richmond, British Columbia, Canada). Packaging plasmids pMDLg/pRRE (Addgene plasmid #12251) and pRSV-Rev (Addgene plasmid #12253) were provided by Didier Trono.

Lentiviral vector design, production, and titration Mutant Sindbis pseudotyped lentiviruses (SINV-LV) were generated via four plasmid transfection in 293T packaging cells. The mutant Sindbis envelope plasmid was constructed by cloning the Sindbis virus glycoprotein insert from plasmid 2.2 (Addgene plasmid no. 34885) into an expression vector plasmid backbone under the CAG promoter. The ZZ domains of Protein A were removed from the mutant E2 domain of the new mammalian expression plasmid via Gibson Assembly cloning. Negative control envelope plasmids for antibody binding specificity studies were kind gifts of Bob Weinberg (pCMV-VSV-G, Addgene plasmid no. 8454) and Jakob Reiser (pCG-HcΔ18, Addgene plasmid no. 84817). To generate functional pseudotyped LV vectors with measles virus glycoproteins, Jakob Reiser also provided the sequence for cloning the measles virus fusion (F) protein envelope plasmid (pCG-FcΔ30). The pLL3.7 transfer plasmid (Addgene plasmid no. 11795) was a gift from Luk Parijs and used as the transgene cassette for expressing eGFP as a reporter of transduction in SINV-GFP. Using Nod and BspEI in a restriction enzyme double digest, we generated a new transfer plasmid, pLL CD19 CAR, from the pLL3.7 plasmid backbone for producing SINV-CAR. The new gene cassette for pLL CD19 CAR consisted of an EF-1α internal promoter, anti-CD19 scFv, CD8 flexible hinge domain, CD8 transmembrane domain, CD28 costimulatory endodomain, CD3C chain, and WPRE post-transcriptional regulatory element all flanked by the original LTRs of the pLL3.7 plasmid backbone. Third generation lentiviral packaging plasmids pMDLg/pRRE (Addgene plasmid no. 12251) and pRSV-Rev (Addgene plasmid no. 12253) were gifts of Didier Trono.(30)

LV were produced via transient transfection of LV-MAX cells according to manufacturer protocols for the LV-MAX lentiviral production system kit (Gibco). Briefly, 1.2×10⁸ viable cells were seeded in a vented shaker flask for a final production volume of 30 mL. A 3:2 ratio of packaging plasmids (envelope, gag/pol, and rev) to transfer plasmid was combined with LV-MAX Transfection Reagent in serum-free medium and subsequently added to cells in shaker flask after 10 minutes of incubation. At ˜48 hours following transfection, cells were collected from suspension culture along with their medium and centrifuged at 1,300×g for 15 mins to pellet cells. Supernatant containing LV vectors was harvested and filtered through a 0.45 μm low protein binding filter to further remove cell debris. Filtered supernatant was added carefully dropwise to a sucrose cushion (25% w/v sucrose in HEPES-NaCl buffer) and subjected to ultracentrifugation at 36,000 rpm for 2.5 hrs at 4° C. Following ultracentrifugation, supernatant and sucrose cushion were carefully aspirated leaving LV pellet at bottom center of tubes. LV pellets were resuspended overnight at 4° C. with 10% w/v sucrose in HEPES-NaCl buffer, aliquoted, and frozen at −80 C for long-term storage. In vivo grade LV was prepared by the Duke University Viral Vector Core (Boris Kantor Lab) using calcium phosphate-based transfection of adherent HEK-293T cells and subsequent double-sucrose gradient purification. All LV were tittered immediately after thawing a fresh aliquot on ice using a qPCR lentiviral titration kit according to manufacturer protocols (Applied Biological Materials Inc., Cat #LV900).

Bispecific antibody construction, expression, and characterization Sequences for chimeric anti-Sindbis E1 or E2 and anti-HER2 antibodies (Ab) were generated by combining the V_{H}/V_{L} regions of commercially available humanized anti-HER2 (Trastuzumab) and murine anti-Sindbis with the C_{H}1/C_{L} and Fc regions of human IgG₁ Ab. Mouse anti-Sindbis E1 and E2 V_{H}/V_{L} sequences were provided by Diane Griffin (Johns Hopkins University; unpublished results). To generate bispecific IgG antibodies (bsIgG₁) that recognized both Sindbis E1 or E2 and anti-HER2, separate orthogonal mutation sets were incorporated into anti-HER2 and anti-Sindbis Fab domains. Orthogonal mutation sets provided high fidelity pairing of heavy and light chains. These mutations were also incorporated into the chimeric monoclonal antibody, IgG₁^{HER2}.

Heavy and light chain antibody constructs were generated on separate mammalian expression vectors, each with the same backbone and CAG promoter sequence. Twist Bioscience performed the molecular cloning of antibody gene constructs for mammalian expression. Following an albumin signal peptide for protein secretion, the bispecific antibody (BsAb) tandem Fab (tFab) heavy chain construct consisted of a murine anti-Sindbis E2 variable heavy domain (V_{H}) and human IgG₁ constant heavy 1 domain (CHI) covalently linked with a humanized anti-CD3 V_{H} and human IgG₁ C_{H1} by a flexible glycine-serine peptide linker (G₄S)₆. The C-terminus of this V_{H}-C_{H1}-Linker-V_{H}-C_{H1} bispecific heavy chain construct contained an 8× polyhistidine tag for purification purposes. A separate construct was designed for each of the two different light chains of the tFab. The anti-Sindbis E2 light chain consisted of a variable light domain and human constant lambda light chain domain (V_{L}-C_{λ}), while the anti-CD3 light chain consisted of a variable light domain and human constant kappa light chain domain (V_{L}-C_{κ}). The murine anti-Sindbis E2 V_{H}/V_{L} sequences were kindly provided by Diane Griffin (Johns Hopkins University; unpublished results), and the anti-CD3 V_{H}/V_{L} sequences were publicly available from a humanized version of the mAb clone UCHT1. To generate the bispecific tFab (Fab^{α-E2}-Linker-Fab^{α-CD3}), separate orthogonal amino acid mutation sets were incorporated into the separate anti-E2 and anti-CD3 Fab domains. These orthogonal mutation sets provided high-fidelity pairing of antibody heavy and light chains for correct assembly of desired BsAb molecule. This OrthoMab technology to generate high-fidelity BsAbs was licensed through a partnership between Dualogics and UNC-CH. A humanized anti-CD3 IgG₁ mAb (IgG₁^{α-CD3}) was also generated with the same set of orthogonal mutations from the tFab's anti-CD3 portion and used as a control molecule for in vitro experimentation.

Plasmids encoding chimeric heavy and light chains were cotransfected into Expi293F cells (Thermo Fisher Scientific, Grand Island, N.Y.) using the ExpiFectamine 293 transfection kit based on manufacturer protocols (Gibco) and grown. IgG₁^{HER2}, bsIgG₁^{E2×HER2}, and bsIgG₁^{E1×HER2} were purified after 72 hours from expression supernatant using protein A agarose (Thermo Fisher Scientific). BsIgG₁ antibodies were separated via size exclusion chromatography (ENnrich SEC 650 10×300 column, Bio-Rad Laboratories, Inc., Hercules, Calif.). The tandem Fab was designed to include a polyhistidine tag on its C-terminus and was purified from expression supernatant using Ni-NTA agarose (Qiagen Inc, Germantown, Md.). tFab required co-transfection of three separate plasmids at equimolar ratios (heavy chain plasmid, anti-E2 light chain plasmid, and anti-CD3 light chain plasmid), while IgG₁^{α-CD3} only required co-transfection of two separate plasmids at equimolar ratios (anti-CD3 heavy chain plasmid including an IgG₁ Fc and anti-CD3 light chain plasmid). After ˜5 days of recombinant protein expression, suspension cells were pelleted by centrifugation at 8,000×g, and the supernatant containing expressed antibodies was harvested and filtered through a 0.2 μm PEG filter. tFab^{α-CD3×α-E2} was purified from cell culture supernatant via immobilized metal affinity chromatography (IMAC) using Ni-NTA agarose (Qiagen). IgG₁^{α-CD3} was purified from cell culture supernatant via affinity chromatography using protein A plus agarose (ThermoFisher Scientific).

Purified proteins were simultaneously concentrated and buffer exchanged into PBS using ultrafiltration (MWCO 30 K, Amicon Ultra). Antibody concentration was determined by spectrophotometry measurements using calculated protein extinction coefficients (A280 NanoDrop™ One/One©). The size and purity of purified antibodies were assessed by sodium dodecyl sulfate polyacrylamide gel electrophoresis (SDS-PAGE), and protein bands were detected with Coomassie stain (Imperial Protein Stain, Thermo Scientific).

Antibody binding assays HER2-specific ELISAs were performed to confirm binding of purified antibodies to HER2 as well as compare dissociation constants of bispecific antibodies relative to parental monoclonal control, IgG₁^{HER2}. Briefly, recombinant human ErbB2/HER2 Fc chimera protein (R&D Systems, cat no. 1129-ER, Minneapolis, Minn.) was coated onto high-binding half-area 96-well Costar plates (Corning) at 1 μg/ml in bicarbonate buffer overnight at 4° C. After blocking plate with 5% nonfat milk in PBS with 0.05% Tween (PBST), purified antibody samples were diluted in 1% nonfat milk in PBST at various concentrations and incubated for 1 h, followed by washes with PBST. Bound antibodies were detected using goat anti-human kappa light chain HRP (Sigma-Aldrich, cat no. A7164, 1:10,0000 dilution) for 1 h followed by 1-step Ultra TMB (Thermo Fisher Scientific). After stopping the HRP reaction with 2N sulfuric acid, the absorbance at 450 nm and 570 nm was measured using a Spectramax M2 plate reader (Molecular Devices).

Indirect enzyme-linked immunosorbent assay (ELISA) was used to characterize and compare binding affinities of purified antibodies to both target antigens: human CD36 and mutant Sindbis E2 glycoprotein. Briefly, either human CD3ε protein (Novus Biologicals, Cat #NBP2-22752) or SINV-LV particles, purified from in-house recombinant production (see above), were coated as antigen onto high binding, half-area, clear 96-well plates (Corning Costar, Cat #3690) overnight at 4° C. Human CD3ε protein was diluted to 1 μg mL⁻¹ in carb-bicarb buffer (pH 9.6, Sigma C3041) for overnight coating, while purified SINV-LV stocks were diluted 100-fold in the same carb-bicarb buffer for overnight coating. The next morning, plates were washed 5× with PBS-0.05% Tween (PBST) and subsequently blocked for 1-2 hours at room temperature with 5% w/v non-fat milk in PBST. Purified antibody samples and controls were serially diluted in 1% w/v milk-PBST, spanning at least three orders of magnitude in concentration, and added to the blocked plates for 1-2 hour incubation at room temperature. Following 5×PBST washes of the plates, bound antibodies were detected using goat anti-human kappa light chain HRP conjugated secondary antibody (Sigma-Aldrich, Cat #A7164) at 1:1,000 dilution in 1% w/v milk-PBST for 1 hour incubation at room temperature. Following 5×PBST washes to remove unbound secondary detection antibody, 1-Step Ultra TMB-ELISA substrate solution (Thermo Scientific) was added for up to 10 mins to detect HRP activity. The enzymatic reaction was quenched by adding equal volume of 2 N sulfuric acid, and the color development was immediately determined by taking absorbance measurements at 450 nm (signal) and 570 nm (background) wavelengths using a SpectraMax M2 microplate reader (Molecule Devices). Negative control wells, including antigen coated, blocked wells without primary antibody incubation and uncoated, blocked wells with primary antibody incubation, both revealed negligible signal development in the assay. Background subtracted absorbance values for each sample condition, run in triplicate, were imported into GraphPad Prism 8 software for calculating the binding affinity of each antibody titration curve and presented as equilibrium dissociation constants (K_{D}). A nonlinear curve fit with one site-specific binding was used to determine the K_{D} values.

To evaluate the specificity of tFab binding to mutant Sindbis glycoproteins, purified LVs, made from the same passage of LV-MAX packaging cells, with different envelopes (SINV, VSV-G, and Measles) were blotted directly onto a nitrocellulose membrane for dot blot immunoassay. Briefly, nitrocellulose membranes were blotted directly with 1 μL of purified LV samples of different envelope pseudotypes. Once samples were dry, the membranes were washed 5× with PBST before blocking the membranes for 1 hour at room temperature in 5% w/v milk-PBST with gentle agitation. IgG1^{α-CD3} negative control or tFab^{α-CD3×α-E2} were diluted separately to 3 μg mL-1 concentration in 1% w/v milk-PBST. The blocked membranes were transferred separately to these primary antibody solutions and incubated for 1 hour at room temperature with gentle agitation for antibody binding. After 5× washes with PBST, primary antibodies bound to the membranes were detected using goat anti-human kappa light chain HRP conjugated secondary antibody (Sigma-Aldrich, Cat #A7164) at 1:1,000 dilution in 1% w/v milk-PBST for 1 hour incubation at room temperature with gentle agitation. After 5× washes with PBST, the membranes were imaged together with identical exposure times using a ChemiDoc XRS+ imaging system (Bio-Rad). Chemiluminescent signal of secondary antibody binding was detected using Clarity Western ECL substrate (Bio-Rad, Cat #1705061).

Viral infectivity assay SKBR3 (HER2⁻) and A2780 (HER2⁻) cells were seeded at 3×10⁴ cells per well in 96-well tissue culture treated plate. Sindbis pseudotyped lentiviruses (multiplicity of infection, MOI=3) were premixed with antibodies at 1 nM concentration for 1 h at room temperature, and then incubated with cells at 37° C. in 5% CO₂. Twenty-four hours later, the transduction mixture was removed from cells and cells were washed three times with PBS. Cells were allowed to grow for 72 h in fresh cell culture media at 37° C. in 5% CO₂. Cells were washed and the percentage of transduced cells (GFP⁺) in each well was quantified using iQue Screener PLUS flow cytometer (Intellicyt, Albuquerque, N. Mex.). Additionally, to confirm that viral infectivity was dependent upon HER2 specificity of the bsAb, the viral infectivity assay was repeated with increasing concentrations of bsIgG₁^{E2×HER2} in the presence and absence of excess IgG₁^{HER2} (100 nM).

To test the selectivity of targeted viral systems for HER2⁺ cells, a co-culture model of SKBR3 and A2780 cells that were maintained in McCoy's 5A medium supplemented with 15% FBS was established. Cells in the co-culture were infected with nontargeted or redirected LV vectors as described above. Seventy-two hours post-infection, treated cells were washed and labeled with IgG₁^{HER2} followed by goat anti-human IgG-Alexa Fluor 594 (Thermo Fisher Scientific) to generate two key cell populations: cells double positive for GFP and HER2 expression and cells double negative for GFP and HER2 expression. The percentages of GFP+ cells of all HER2⁺ cells and GFP⁺ cells of all HER2⁻ cells in each well were quantified using iQue Screener PLUS flow cytometer. Data were analyzed using ForeCyt software and BD FACSDiva software.

In vitro transduction assays The CD3⁺ Sup-T1 tumor cell line was transduced with SINV-GFP in the presence of increasing concentrations of tFab to demonstrate BsAb-mediated enhanced transduction of target cells. Sup-T1 cells were seeded in sterile 96-well tissue culture treated plates (Corning Costar Cat #3599) at 1×10⁵ cells/well. SINV-GFP at a multiplicity of infection (MOI) of 25, based on qPCR tittering, was premixed with various concentrations of tFab in serum-containing growth culture medium for 1 hour at room temperature to allow tFab to bind onto the surface of SINV-GFP particles before directly adding this transduction mixture to the plated cells. Each tFab concentration tested (1, 10, 30, and 50 nM) is reported as the final concentration of the tFab once diluted and added to cells for transduction in 96-well plates. To confirm enhanced transduction was dependent on the CD3 specificity of the tFab, excess IgG₁^{α-CD3} (300 nM) was added to replicate sample wells at each tFab concentration to competitively block binding of CD3 as entry receptor for targeted transduction with SINV-GFP plus tFab. After 24 hours of transduction at 37° C. and 5% CO₂, cells were washed twice with cold growth culture medium using low-speed plate centrifugation (300×g) to remove residual antibody and LV prior to resuspension in fresh growth culture medium. Cells were allowed to grow and express GFP transgene for 72 hours at 37° C. and 5% CO₂ prior to washing them into PBS and analyzing their GFP expression via flow cytometry using an Attune N×T flow cytometer with plate autosampler (Applied Biosystems).

A similar transduction assay with CD3+ Sup-T1 and CD3− BV-173 tumor cell lines was established to demonstrate specificity and selectivity of SINV-GFP plus tFab transduction to CD3⁺ target cells. Sup-T1 and BV-173 cells were seeded together at a 1:1 ratio in each well of sterile 96-well tissue culture treated plates (Corning Costar Cat #3599) at 1×10⁵ total cells/well. SINV-GFP at a MOI of 25, based on qPCR tittering, was premixed with 30 nM final concentration of tFab in serum-containing growth culture medium for 1 hour at room temperature before directly adding this transduction mixture to the plated co-culturing cells. A control transduction of SINV-GFP at MOI 25 without addition of tFab was also dosed to co-culturing cells. After 24 hours of transduction at 37° C. and 5% CO₂, cells were washed twice with cold growth culture medium using low-speed plate centrifugation (300×g) to remove residual antibody and LV prior to resuspension in fresh growth culture medium. Cells were allowed to grow and express GFP transgene for 72 hours at 37° C. and 5% CO₂ prior to washing them into PBS for surface marker phenotype staining with anti-CD3 APC (BD Cat #340440) and anti-CD19 PE (BD Cat #340364). Phenotypic antibody staining was allowed to proceed for 30 mins at 4° C. followed by two PBS washes of samples to remove unbound antibodies. Washed cells were resuspended into PBS and analyzed for their GFP expression via flow cytometry using an Attune N×T flow cytometer with plate autosampler (Applied Biosystems).

Activated primary human PBMCs were transduced with SINV-CAR at a MOT of 10, based on qPCR, with and without addition of tFab to demonstrate functional CAR expression and subsequent cytotoxic activity of CAR-T cells in vitro. In brief, 2.5×10⁵ activated PBMCs were transduced in 250 uL final volume per well of growth culture medium supplemented with IL-7 and IL-15 cytokines in 48-well tissue culture treated plates. SINV-CAR at a MOI of 10, based on qPCR tittering, was premixed with 50 nM final concentration of tFab in serum-containing growth culture medium for 1 hour at room temperature before directly adding this transduction mixture to the plated PBMCs. SINV-CAR at MOI 10 was also dosed directly without addition of tFab for targeting along with other non-transduced control PBMC sample wells. After 6 hours of transduction at 37° C. and 5% CO₂, PBMC samples were washed twice with cold growth culture medium to remove residual antibody and LV prior to resuspension in fresh growth culture medium and transfer to a new, sterile 24-well tissue culture treated plate for 84 hours of growth and CAR expression at 37° C. and 5% CO₂. A portion of each sample well was collected and washed into PBS for phenotypic surface marker staining by a panel of antibodies and subsequent CAR expression analysis using an LSR Fortessa flow cytometer (BD Biosciences). The remaining PBMCs in each sample well were resuspended and counted by trypan blue dye exclusion for subsequent plating with CD19⁺ tumor B cells to demonstrate CAR functionality by a co-culture cytotoxicity assay described in more details below.

In vitro co-culture tumor cytotoxicity assay Transduced and non-transduced control PBMCs (1.5×10⁵ cells/well or 3×10⁵ cells/well) were cocultured with tumor cell lines (BV-173 or Daudi, 1.5×10⁵ cells/well in 24-well plates), in complete medium, in the absence of cytokines (E:T=1:1 or E:T=2:1). The effector-to-target (E:T) ratio was not corrected for the percentage of CAR⁺ T cells but was calculated based on the total number of T cells in culture. After 4-5 days of culture, cells were harvested and stained with CD3 (APC-H7, clone SK7 from BD Biosciences) and CD19 (FITC, clone SJ25C1 from BD Biosciences) monoclonal Abs to detect T cells and tumor cells, respectively. Residual tumor cells in culture were enumerated by flow cytometry. Culture supernatants were harvested after 24 or 48 hours of culture and IFN-γ and IL-2 measured using the DuoSet Human IFN-γ and DuoSet Human IL-2 ELISA kits respectively (R&D Systems). Data acquisition was performed on a Synergy2 microplate reader (BioTek) using the Gen5 software.

Tumor mouse model for testing efficacy of in vivo generated CAR-T cells All tumor mouse model experiments were performed in accordance with UNC Animal Husbandry and Institutional Animal Care and Use Committee (IACUC) guidelines and were approved by UNC IACUC (Protocol #: 18-251). Female NSG mice (7-9 weeks of age, obtained from the UNC Animal Services Core) were used to establish the chronic myeloid leukemia xenograft tumor mouse model. Mice were irradiated at a low dose (100 rad) by a cesium irradiator on Day −6 of the study prior to any cell engraftments. The following day (Day-5), 5×10⁵ FFLuc BV-173 tumor B cells were injected in 150 μL sterile PBS via i.v. tail vein. After allowing 5 days for tumor cell engraftment, 5×10⁶ activated PBMCs were injected on Day 0 in 150 uL sterile PBS via i.v. tail vein. 30 minutes after infusing the PBMCs, mice were randomly separated into two different treatment groups: (1) SINV-CAR without tFab or (2) SINV-CAR with premixed tFab. In both groups, SINV-CAR was dosed at 2.5×10⁷ infectious units (IU), based on qPCR, in 150 uL sterile PBS per mouse via i.v. tail vein injection. This dosage equated to 5×10¹⁰ viral particles per mouse, based on absolute particle counts of SINV-CAR using NanoSight NS500 (Malvern Panalytical) nanoparticle tracking analysis. tFab (5 μg/mouse) was premixed with SINV-CAR for 1 hour at room temperature in 150 μL sterile PBS prior to i.v. injections. B cell tumor growth was monitored weekly by bioluminescent imaging (BLI; total flux, photons/second) using an Ami HT optical imaging system (Spectral Instruments Imaging). Peripheral blood samples were taken weekly from mice via the submandibular route. Peripheral blood was subjected to red blood cell lysis followed by antibody staining and flow cytometry to assess number of human T cells (CD3⁺) and tumor B cells (CD19⁺) in circulation. Mice were sacrificed according to UNC guidelines for either tumor growth or occurrence of signs of discomfort, such as tumor-mediated paralysis. Upon sacrifice, peripheral blood was collected from cardiac puncture of the heart, and spleens were measured and weighed prior to smashing over cell strainers into single cell suspensions. Blood and spleen were subjected to red blood cell lysis, antibody staining, and flow cytometry using an LSR Fortessa flow cytometer (BD Biosciences) to detect and quantify CAR⁺ T cells and CD19⁺ tumor B cells in isolated tissues. Antibodies used for phenotypic staining of in vivo samples included CD3 (APC-H7, clone SK7), CD8 (Alexa Fluor 700, clone RPA-T8), CD45 (APC, clone 2D1) and CD19 (FITC, clone SJ25C1) along with CountBright absolute counting beads (Invitrogen). All flow cytometry data analysis was performed with FlowJo v10 software.

Immunophenotyping T cells were stained with Abs against CD3 (APC-H7, clone SK7), CD8 (Alexa Fluor 700, clone RPA-T8) and CD45 (APC, clone 2D1) from BD Biosciences. Tumor cells were stained with Ab against CD19 (FITC, clone SJ25C1) from BD Biosciences. The expression of the anti-CD19 CAR was assessed using specific anti-idyotipic Ab, followed by the staining with a secondary rat anti-Mouse Ab (PE, clone X56) from BD Biosciences. Data acquisition was performed on BD LSRFortessa or Canto H flow cytometer using the BD FACS-Diva software or on a MACSQuant (Miltenyi Biotec). Data analyses was performed with the FlowJo software (Version 9 or 10).

Transmission electron microscopy (TEM) of lentivirus Purified SINV-LV was incubated on a glow discharged CF300Cu grid. Excess sample was wicked away from the grid and rinsed with washing buffer (lx PBS). The grid was blocked in 1% w/v BSA-PBS, rinsed with washing buffer, and incubated with tFab (10 μg mL⁻¹) at room temperature. Following another buffer rinse, secondary gold bead conjugated antibody (Abcam, Cat #ab39596) was incubated with the grid at a final stock dilution of 1:50 at room temperature. The grid was rinsed with washing buffer prior to addition of 4% PFA for fixation. Following a final buffer rinse, negative staining was performed. The grid was rinsed with DI water followed by addition of 1% uranyl acetate solution to the grid for 10 minutes. A final rinse with DI water was performed. The entire process took place in a 150×15 mm petri dish to prevent evaporation of solutions. Images were captured using an FEI Tecnai T12 transmission electron microscope at 120 kV.

Statistical analysis All data are presented as mean±SD. All graphs and statistical tests were performed using GraphPad Prism 7 or 8 software. Either a post hoc Tukey's test or Bonferroni correction was performed to correct for multiple comparisons after two-way ANOVA. Survival analysis was performed using the Kaplan-Meier method with a log-rank test to determine statistical significance. All p values less than 0.05 were considered statistically significant.

### Example 1

### OrthoMab-Based Bispecific Antibodies (bsAbs) Preserve Specificity and Affinity to Antigens

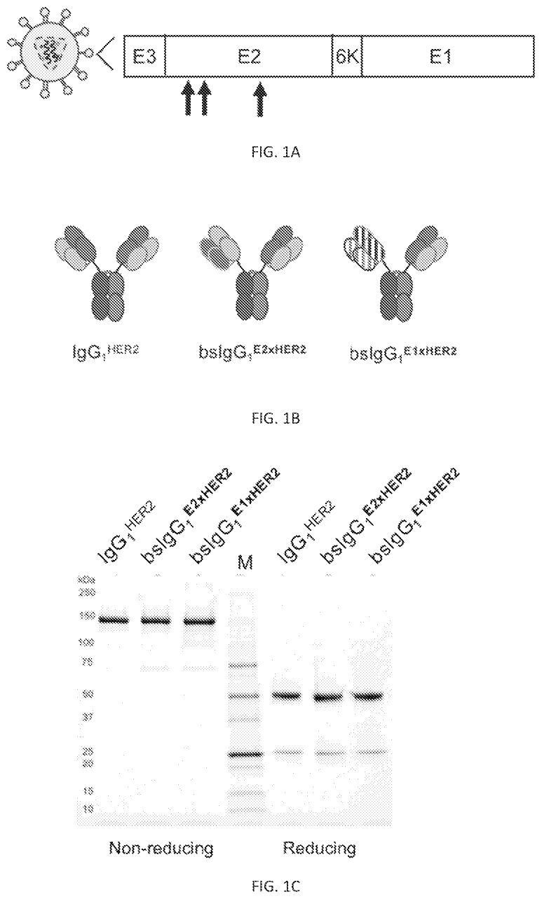

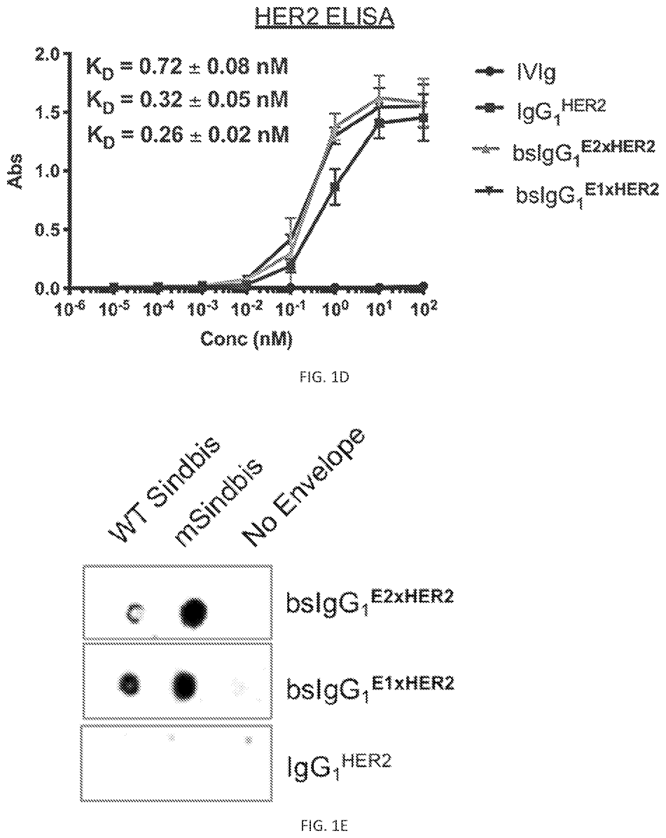

A chimeric bsAb was engineered against both (i) HER2 overexpressed on breast cancer cells and (ii) Sindbis Env glycoproteins displayed on LV. This was accomplished by merging human IgG1 backbones with HER2⁻ and Sindbis envelope-binding V_{H} and V_{L} domains previously isolated from mouse IgG. Bispecific antibodies were prepared that bound either Sindbis Env glycoprotein E1 (responsible for pH-dependent endo-lysosomal membrane fusion and escape) or E2 domain (responsible for binding high-affinity laminin receptors or heparin sulfate for cellular entry) (FIG. 1A). Purified bsAb were separated via size exclusion chromatography, and exhibited the expected molecular sizes as visualized on non-reduced and reduced protein gels (FIGS. 1B-C).

The specificity and affinity of the bsAb was confirmed using antigen-specific ELISAs against HER2. Both bispecific bsIgG₁^{E2×HER2} and bsIgG₁^{E1×HER2} possessed similar binding affinities to HER2 as the monoclonal anti-HER2 IgG₁ (Trastuzumab; IgG₁^{HER2} control): the K_{D} for bsIgG₁^{E2×HER2}, bsIgG₁^{E1×HER2}, and IgG₁^{HER2} were 0.32±0.05 nM, 0.26±0.02 nM, and 0.72±0.08 nM, respectively (FIG. 1D). The binding of the bsAb to WT- and mSindbis pseudotyped LV was also assessed using dot blot. Both bsAb bound WT and mSindbis Env pseudotyped LV and did not bind to LV without an envelope (i.e. negative control) (FIG. 1E). As expected, IgG₁^{HER2} did not bind to WT Sindbis, mSindbis, or the non-enveloped LV control. Altogether, these results confirmed a functional bsAb, and that the orthogonal mutations introduced at the heavy and light chain interface for both Fabs did not impair binding to either HER2 or Sindbis envelope.

### Example 2

### bsIgG₁^{E2×HER2} Enhanced Mutated LV Infectivity Compared to Wildtype LV Alone

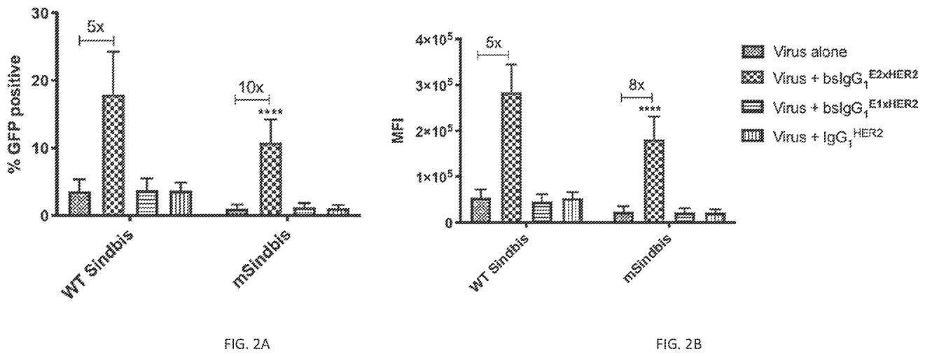

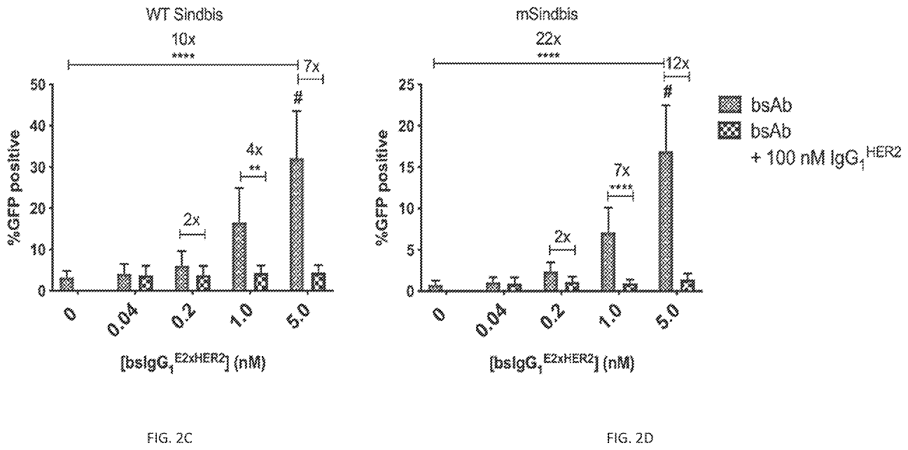

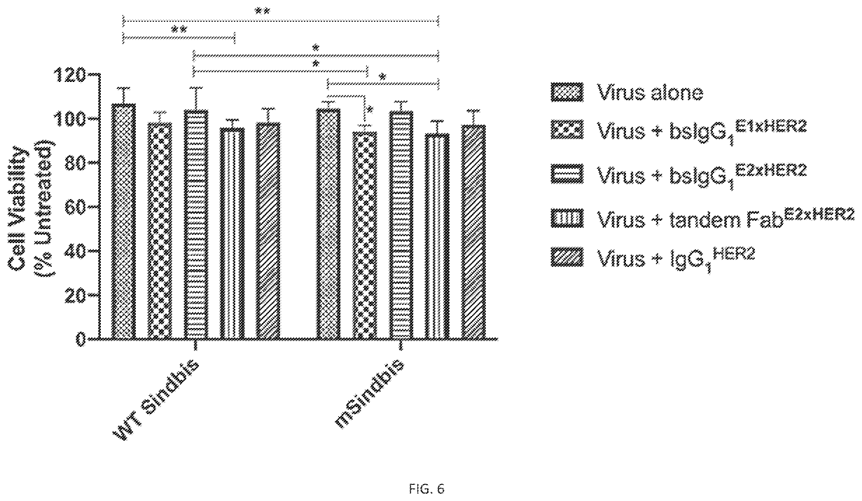

Using flow cytometry, the transduction efficiency of native, nontargeted WT and mSindbis lentiviruses expressing GFP in HER2⁺ SKBR3 cells was measured using a low vector-to-cell ratio (commonly referred to as multiplicity of infection, or MOI) of three. As expected, mSindbis had markedly lower transduction efficiency compared to WT Sindbis, transducing only ˜1% of target HER2⁺ cells vs ˜4% for WT Sindbis, with two-fold lower mean fluorescence intensity (MFI) than WT Sindbis (FIGS. 2A & 2B). The infectivity of both WT and mSindbis LV were both substantially enhanced when pre-mixed with 1 nM of E2-binding bsIgG₁^{E2×HER2}, resulting in transduction of ˜18% and ˜12% of HER2⁺ cells at the same MOI, respectively (FIGS. 2A & 2B). Compared to nontargeted WT Sindbis, the redirected WT Sindbis transduced 5-fold more target cells, with 5-fold greater MFI, whereas redirected mSindbis transduced 10-fold more target cells than mSindbis alone, with 8-fold greater MFI. These results indicated that bsAb can confer greater cell binding of LV, with more pronounced improvement seen for mSindbis versus WT Sindbis, most likely due to the exceedingly limited transduction by mSindbis LV alone. Targeted LV treatment also maintained a similar level of cytotoxicity compared to both untreated cells and cells treated with LV alone, suggesting that lentiviral redirection using bsAB is not toxic to cells (FIG. 6).

Whether increasing the concentration of bsIgG₁^{E2×HER2} could further enhance the transduction efficiency of both redirected LV was assessed next. At the highest bsIgG₁^{E2×HER2} concentration tested, redirected WT Sindbis and mSindbis LV transduced ˜32% and ˜17% of SKBR3 cells, increasing the fraction of GFP⁺ SKBR3 cells by ˜10-fold and ˜22-fold, respectively, compared to their corresponding nontargeted LVs (FIGS. 2C & 2D). BsIgG₁^{E2×HER2} redirection was highly specific to HER2, as incubation with excess IgG₁^{HER2} control effectively blocked transduction, reducing the percentage of GFP⁺ cells at each tested bsAb concentration to the same level as nontargeted LVs (FIGS. 2C & 2D).

To assess whether bsIgG simply need to engage the LV or if efficient transduction is dependent on bsIgG binding to specific viral epitopes, the transduction potencies of LVs pre-mixed with bsIgG1^{E1×HER2} were evaluated in parallel. Interestingly, bsIgG₁^{E1×HER2} did not improve the transduction efficiency of either LV at all, with comparable percentages of GFP⁺ cells and MFI of transduced cells to that of nontargeted LV alone (FIGS. 2A & 2B). Nontargeted WT Sindbis, WT Sindbis mixed with bsIgG₁^{E1×HER2}, and WT Sindbis mixed with IgG₁^{HER2} control all transduced ˜4% of HER2⁺ cells. Similarly, nontargeted mSindbis, mSindbis mixed with bsIgG₁^{E1×HER2}, and mSindbis mixed with IgG₁^{HER2} control all transduced ˜1% of HER2⁺ cells. These results indicated that bsAb-mediated gene transfer by LV is critically dependent on bsAb engaging specific epitopes on the Sindbis Env-binding domain on the LV surface.

### Example 3

### Targeted LV Vectors Preferentially Transduced Target HER2⁺ Cells

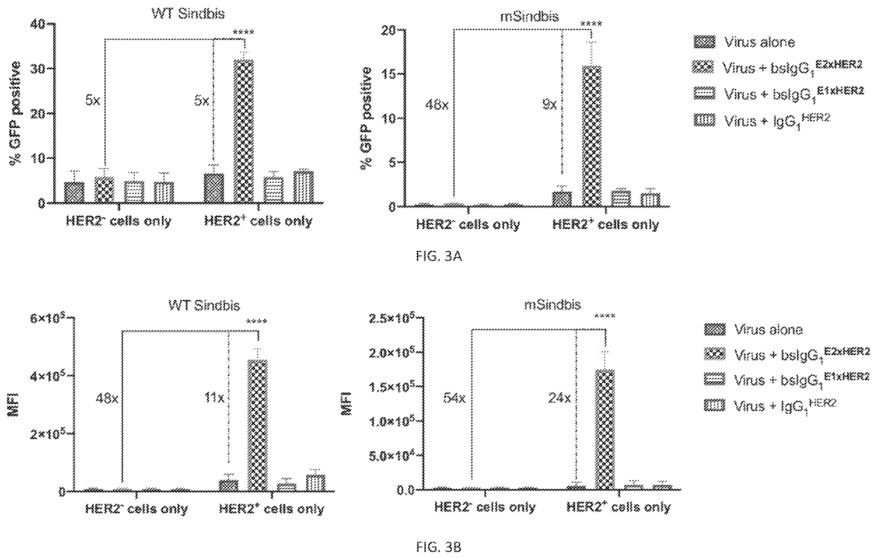

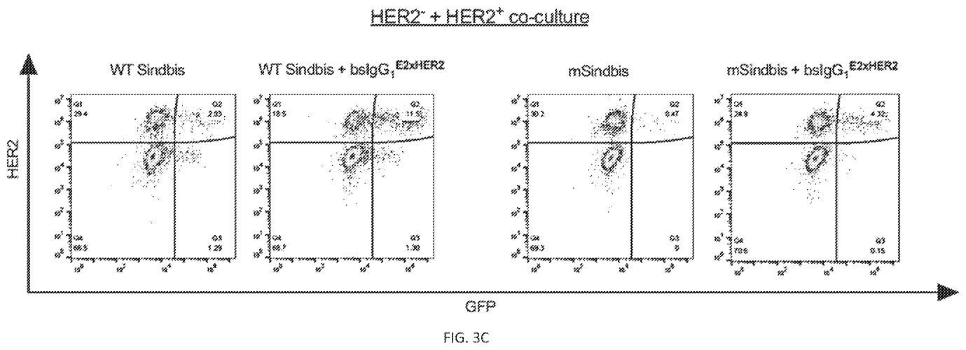

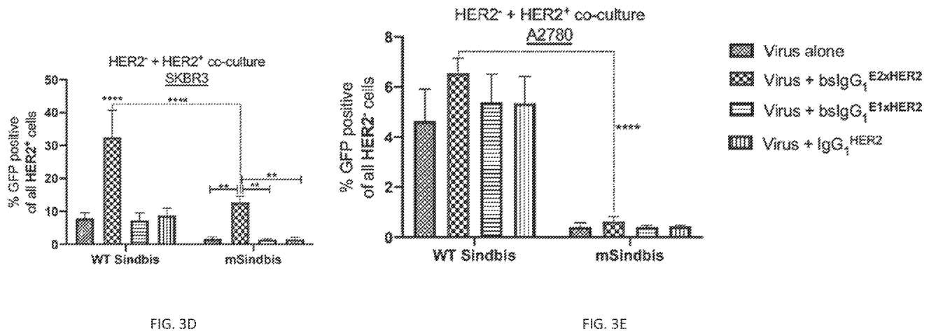

To evaluate the specificity of bsAb-mediated LV for target cells relative to off-target cells, their transduction potencies on HER2⁺ (SKBR3) and HER2⁻ (A2780) cells were separately compared, where A2780 represented a nonspecific cell control with little to no HER2 expression. As expected, a comparable transduction of HER2 cells with either WT and mSindbis LV alone (5% and 0.2% of A2780 cells, respectively) as with HER2⁺ cells (7% and 1.7% of SKBR3 cells, respectively) (FIG. 3A) was observed. Pre-mixing LV with bsIgG₁^{E2×HER2} did not appreciably increase transduction of HER2 cells, with 6% and 0.3% of A2780 cells transduced with redirected WT and mSindbis LV (FIG. 3A). Compared to WT Sindbis LV alone, bsIgG₁^{E2×HER2}-targeted WT Sindbis increased the percentage of GFP⁺ cells by 5-fold (FIG. 3A, dotted line) and MFI by 11-fold (FIG. 3B, dotted line). Redirecting mSindbis LV with bsIgG₁^{E2×HER2} led to greater improvement over mSindbis LV alone, with a 9-fold increase in the percentage of GFP⁺ cells (FIG. 3A, dotted line) and 24-fold higher MFI (FIG. 3B, dotted line). Both redirected LVs demonstrated markedly greater selectivity for HER2⁺ cells over HER2 cells, with redirected mSindbis LV substantially exceeding the specificity of targeted WT Sindbis LV. In particular, WT Sindbis LV redirected with bsIgG₁^{E2×HER2} increased the percentage of GFP⁺ cells by 5-fold (FIG. 3A, solid line) and MFI by 48-fold (FIG. 3B, solid line) in HER2⁺ SKBR3 cells compared to HER2⁻ A2780 cells. Similarly, redirected mSindbis LV transduced 48-fold more SKBR3 cells than A2780 cells, with 54-fold higher MFI than mSindbis LV alone (FIGS. 3A & 3B, solid lines).

To further assess the specificity of gene transfer, bsIgG₁^{E2×HER2}-targeted LV were assessed for selectively transducing HER2⁺ cells in co-cultures of both HER2⁺ and HER2⁻ cells. In good agreement with its broad transduction nature and results from mono-culture experiments, nontargeted WT Sindbis had very poor selectivity, transducing ˜8% of HER2⁺ cells (FIG. 3D) and ˜5% of HER2⁻ cells in this co-culture setting (FIG. 3E). Nontargeted mSindbis LV also had relatively limited selectivity, transducing ˜2% of HER2⁺ cells (FIG. 3D) and ˜0.4% of HER2⁻ cells (FIG. 3E). Redirecting WT Sindbis LV with bsIgG₁^{E2×HER2} modestly increased both the potencies and specificity: targeted WT Sindbis LV exhibited a ˜5× selectivity towards HER2⁺ cells, transducing ˜33% of SKBR3 cells vs ˜7% of A2780 cells (FIGS. 3D & E). In contrast, combining bsAb-based redirection with ablation of native receptor binding synergistically enhanced targeting efficiencies, with a ˜20× selectivity towards HER2⁺ than HER2⁻ cells (˜13% of SKBR3 cells vs ˜0.6% of A2780 cells) in the co-culture study. Overall, compared to WT Sindbis LV alone, redirected mSindbis LV were ˜2-fold more efficient in transducing SKBR3 cells, while reducing non-specific gene transfer by ˜22-fold (˜13% of HER2⁺ cells vs ˜0.6% of HER2⁻ cells). These results underscored the enhanced selectivity and potent gene transfer using mSindbis LV redirected with bsIgG₁^{E2×HER2}.

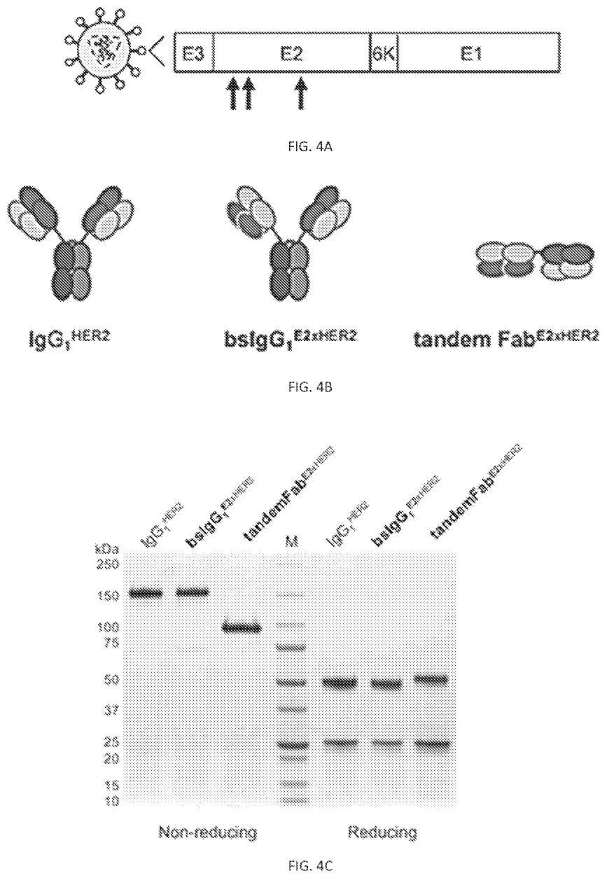

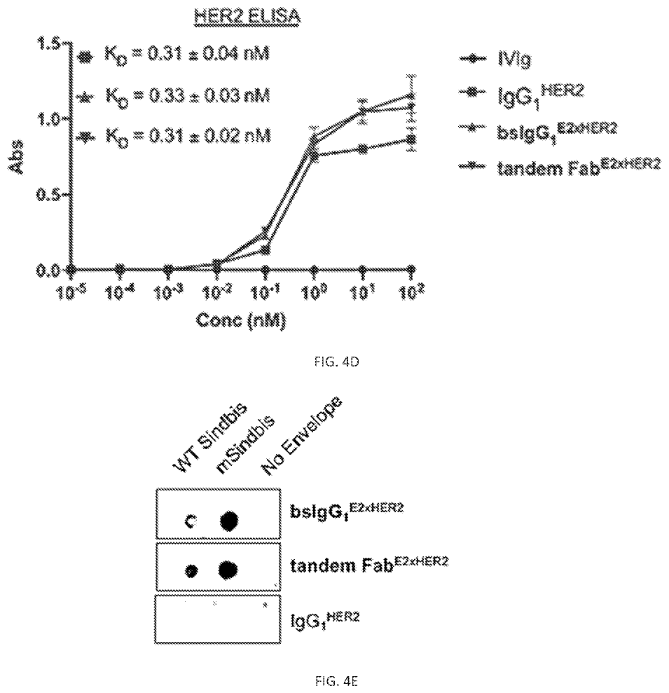

For in vivo applications, FcRn recycling and non-specific uptake by Fc receptors on immune cells present a challenge for in vivo efficiency of targeted viral vectors via systemic administration. A Fc-free tandem Fab that similarly binds Sindbis E2 and HER2 (FIGS. 4A & 4B) was evaluated. The tandem Fab exhibited the expected molecular sizes as visualized on non-reduced and reduced protein gels (FIG. 4C). Using HER2-specific ELISAs, it was found that tandem Fab^{E2×HER2} and bsIgG₁^{E2×HER2} possessed comparable binding affinities to HER2 as the monoclonal IgG₁^{HER2} control (FIG. 4D). Also verified via dot blot was the binding of tandem Fab to WT- and mSindbis-pseudotyped LV, but not envelope-null LV (ie. negative control). The negative antibody control, IgG₁^{HER2}, did not bind to WT Sindbis, mSindbis, or non-enveloped LV (FIG. 4E).

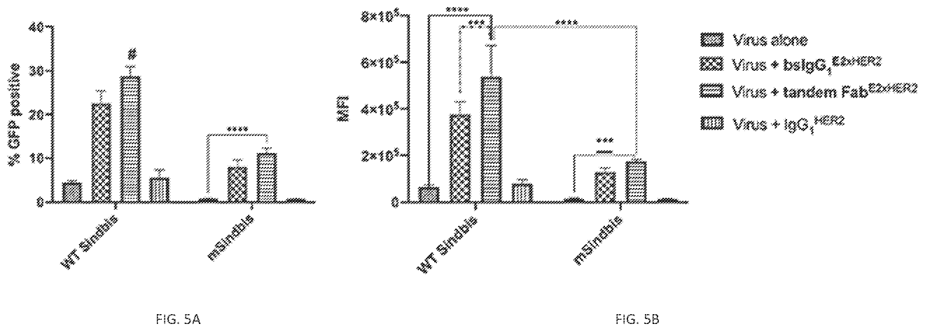

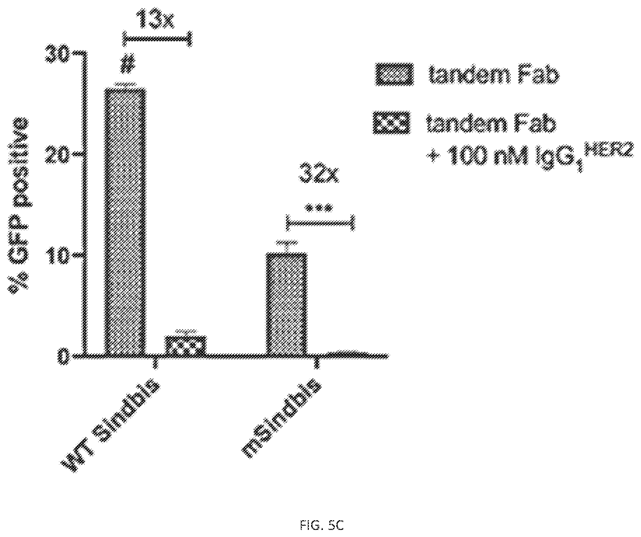

The transduction efficiency of targeted LV with bsIgG₁^{E2×HER2} versus tandem Fab^{E2×HER2} using flow cytometry was compared. As expected, bsIgG₁^{E2×HER2} transduced ˜5-fold more SKBR3 cells compared to WT Sindbis LV, and ˜10-fold vs mSindbis (FIG. 5A, 5B). Tandem Fab^{E2×HER2} also enhanced the transduction efficiency of WT Sindbis and mSindbis by ˜6-fold and ˜14-fold, respectively (FIGS. 5A & 5B). At the tested bsAb concentrations, there was no statistical difference in transduction efficiency when LV were mixed with bsIgG₁^{E2×HER2} or tandem Fab^{E2×HER2}. BsIgG₁^{E2×HER2} and tandem Fab^{E2×HER2} redirection was highly specific to HER2, as incubation with excess IgG₁^{HER2} control efficiently blocked transduction, reducing the percentage of GFP⁺ cells (FIG. 5C). Overall, the tandem Fab facilitated similar transduction effectiveness as bsIgG₁.

### Example 4

### Bispecific Binder Redirected Lentiviral Vector Enables In Vivo Engineering of CAR-T Cells

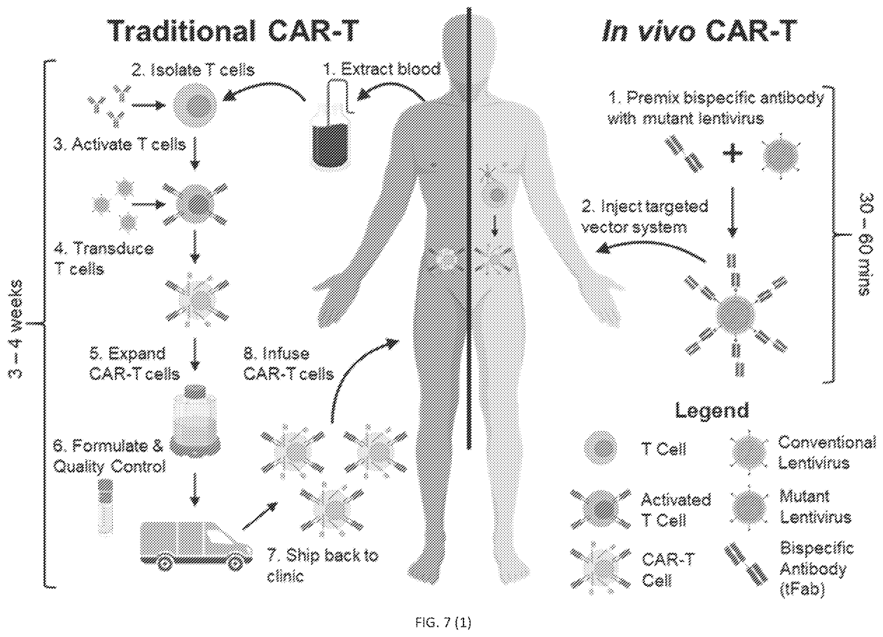

Adoptive transfer of CD19-specific CAR-T cells has demonstrated considerable success for the treatment of B cell malignancies in patients with relapsed or refractory diseases, providing the basis for three cell therapies approved by the U.S. Food and Drug Administration (FDA) to date. However, the generation of CAR-T cell products in all instances involves time consuming and complex manufacturing processes that delay the immediate availability of these cellular therapies for patients with aggressive disease, and also lead to exorbitant costs (FIG. 7-left). Furthermore, activation, genetic manipulation, and ex vivo expansion of CAR-T cells inevitably leads to significant differentiation of T cells, which likely reduce their self-renewal capacity upon adoptive transfer back into patients and consequently limiting the overall efficacy.

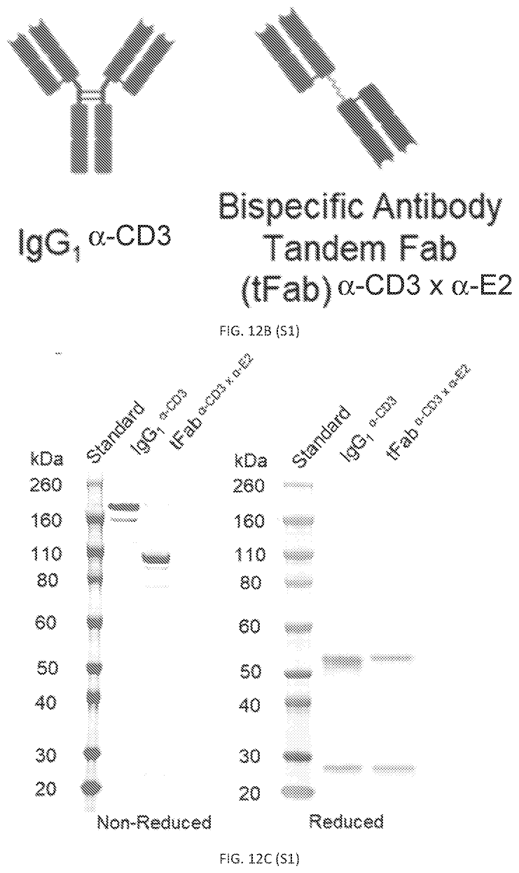

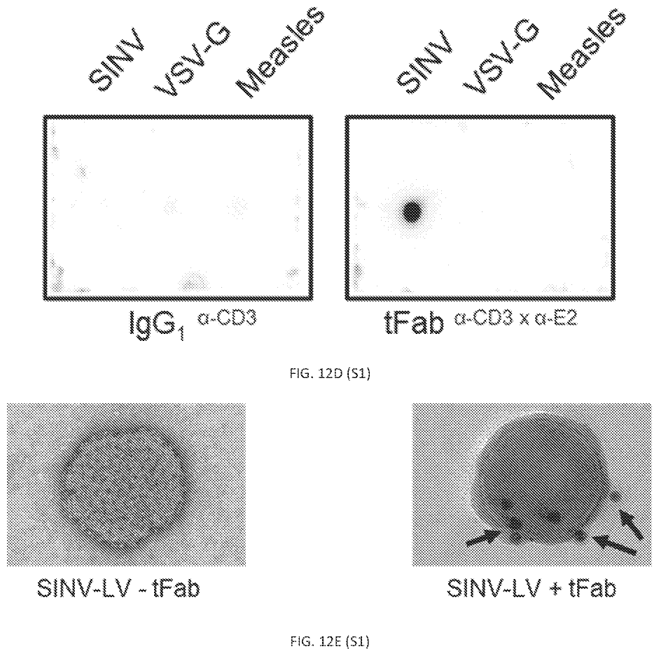

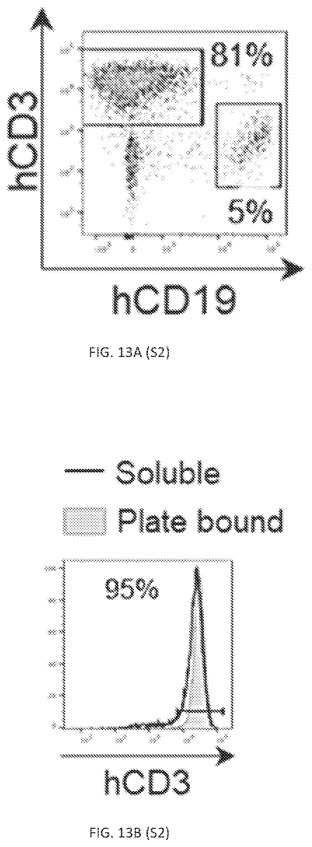

Direct in vivo engineering of CAR-T cells, based on transducing T cells circulating in the peripheral blood with viral vectors as described herein, may bypass the need for ex vivo manufacturing of patient-derived T cells entirely (FIG. 7-right). Herein, a lentiviral-based gene transfer system with considerable specificity and efficiency for T cell targeting in vivo was developed. To minimize transduction of non-target cells, a mutated Sindbis pseudotyped lentiviral vector (SINV-LV) was incorporated with mutations to the E2 glycoprotein that abrogate its native tropism to human cells (FIG. 12A). To redirect the SINV-LV that lacks specific cell tropism to T cells, bispecific binders that can bind: (i) the E2 glycoprotein on SINV-LV and (ii) CD3, a ubiquitous co-receptor on all T cells were engineered.

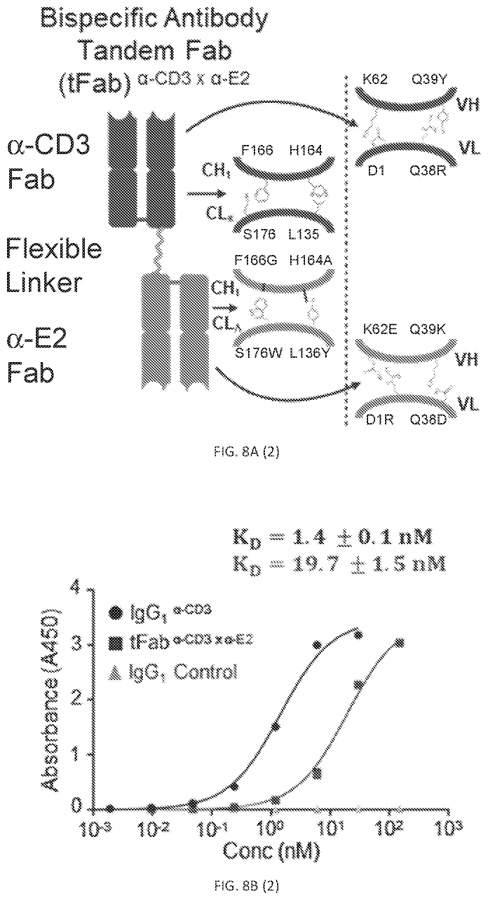

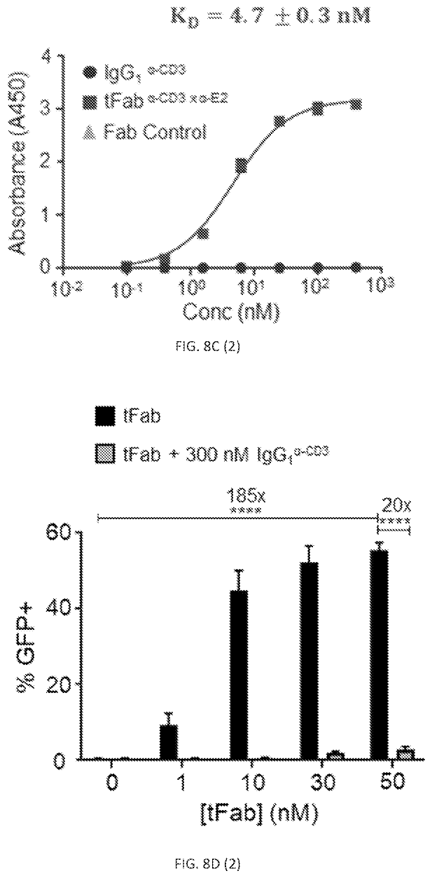

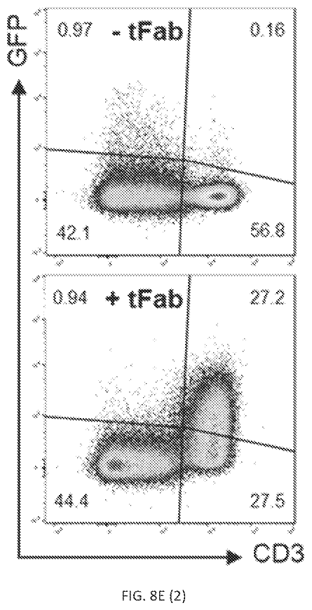

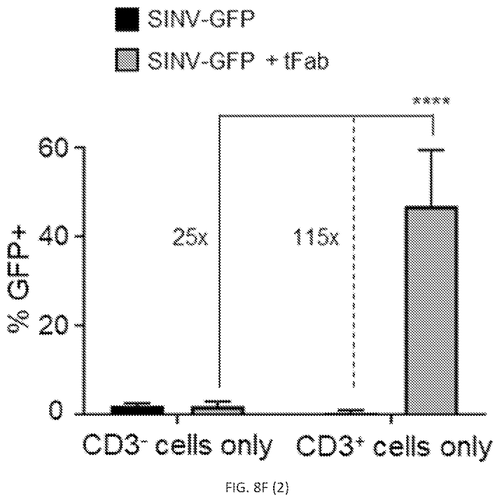

Bispecific binders in a tandem Fab format (tFab), comprised of two distinct Fab domains linked via a glycine-serine flexible linker and lacking the Fc antibody domain (FIGS. 8A & 12B) were engineered. By applying different sets of orthogonal amino acid mutations to the two Fab domains (anti-CD3 and anti-E2), traditional heavy/light chain mispairing was overcome and a pure population of bispecific tFab binders with properly paired Fabs were produced by simple immobilized metal affinity chromatography (IMAC) purification (FIG. 12C). Several immunoassays were performed, including ELISAs (FIGS. 8B & 8C), dot blots (FIG. 12D), and immunogold labeling with transmission electron microscopy (TEM) (FIG. 12E), to characterize the specificity and affinity of the tFab binding to both human CD3ε and mutant Sindbis E2 glycoprotein. The tFab bound to both CD3c and E2 at low nanomolar affinities (K_{D}=19.7 nM and 4.7 nM, respectively) as assessed by ELISA, whereas control anti-CD3 IgG of the same Fab clone (IgG₁^{α-CD3}) bound only to CD3ε. Anti-CD3 IgG possessed higher binding affinity (K_{D}=1.4 nM), which was likely a direct consequence of the dimeric nature of two Fabs per IgG molecule. Using different lentivirus pseudotypes including SINV, VSV-G, and Measles Virus in dot blot experiments, tFab was confirmed to bind specifically to only SINV-LV.

To evaluate the capacity of the SINV/tFab platform in targeting human T cells, SINV-LV encoding an eGFP fluorescent reporter transgene (denoted as SINV-GFP) were generated, mixed with different amounts of tFab, and the level of induced eGFP expression in a CD3 human cell line was quantified. A tFab dose-dependent transduction enhancement saturated at ˜50 nM concentration of tFab (FIG. 8D). Without addition of the tFab, the transduction efficiency of SINV-GFP alone was less than 1%, whereas 50 nM of tFab enabled transduction of >50% of the cells. The increased transduction was a direct consequence of the combination of SINV-GFP and tFab redirection, as demonstrated by competitive inhibition in the presence of excess amounts of anti-CD3 IgG₁ (300 nM) (FIG. 8D). To further validate the specificity of viral targeting, SINV-GFP/tFab was tested in co-culture experiments mixing CD3⁺ and CD3⁻ (BV-173) cells. Without addition of the tFab, SINV-GFP showed negligible transduction of either CD3⁺ or CD3⁻ cells (FIG. 8E). In contrast, SINV-GFP/tFab showed a ˜25-fold enhanced transduction of CD3⁺ vs. CD3⁻ cells (FIG. 8F).

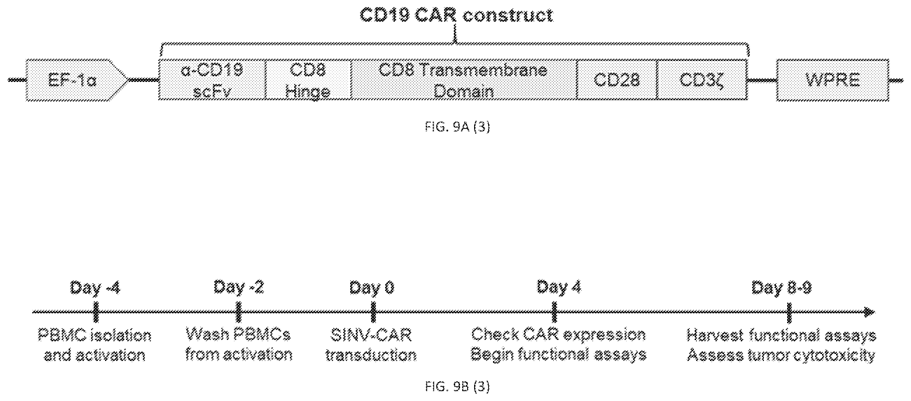

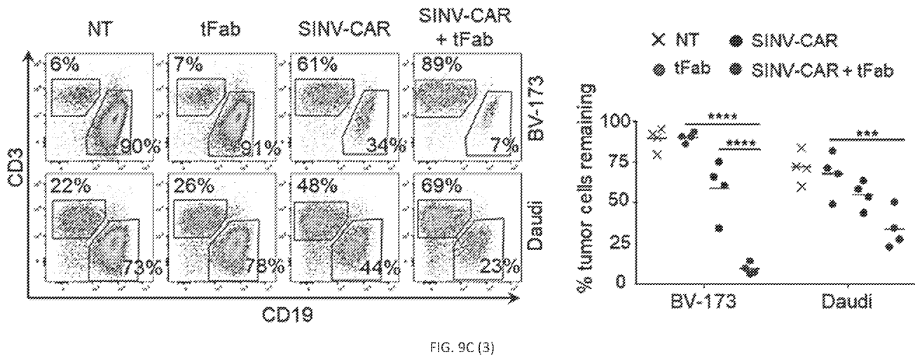

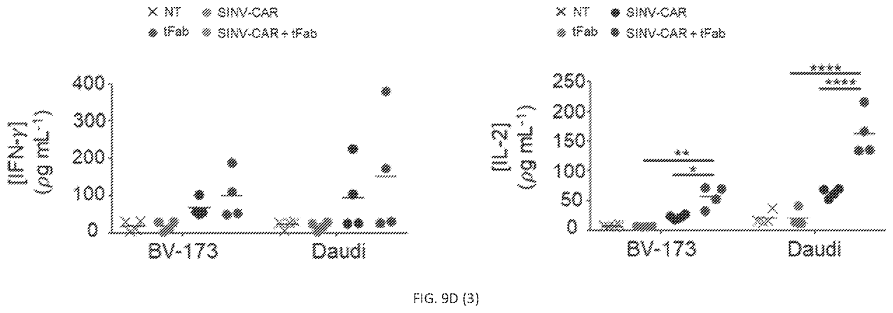

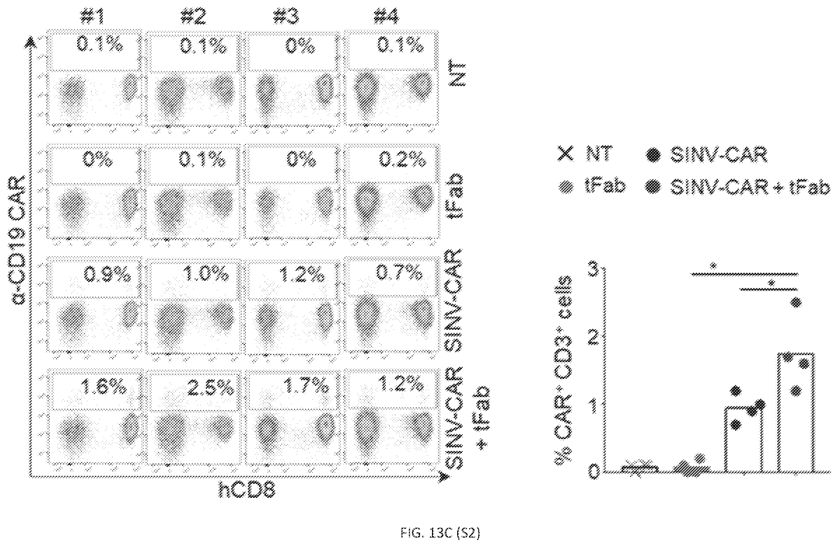

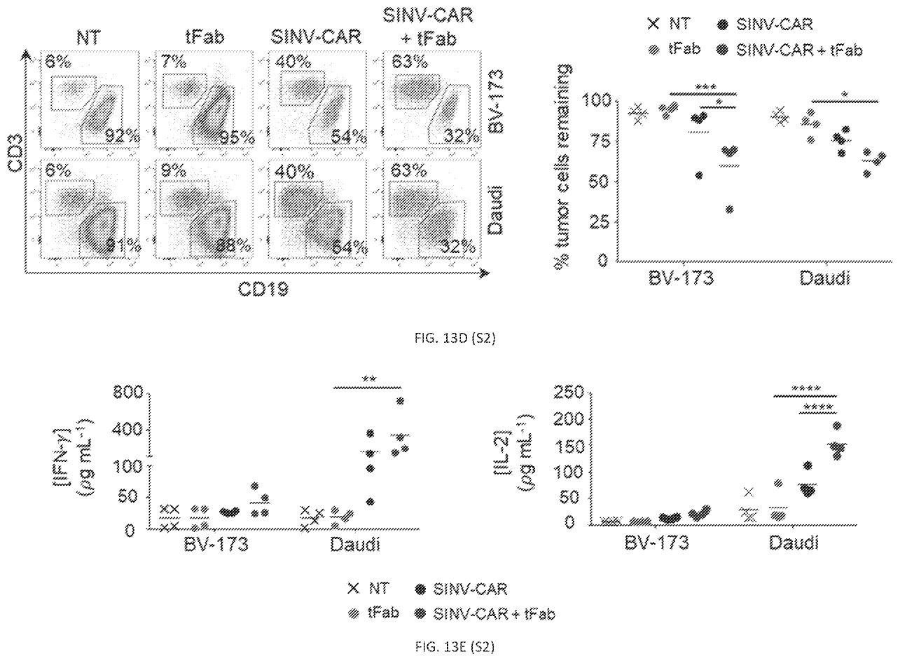

A second-generation CD19-specific CAR encoding the CD28 costimulatory endodomain was cloned into the SINV-LV (SINV-CAR; FIG. 9A) and the transduction efficiency was tested in primary human T cells. At relatively low multiplicities of infection (MOI=10), the SINV-CAR/tFab yielded ˜1.2-2.5% CAR-T cells, including both CD4⁺ and CD8⁺ cells, which was a significantly higher fraction than the SINV-CAR alone (P=0.0437; FIG. 13C). To determine if CAR-T cells were functionally active, an in vitro co-culture assay was developed to measure CAR-T cell cytotoxicity and cytokine secretion in presence of CD19⁺ tumor cells (BV-173) (FIG. 9B). Even at very low effector-to-tumor (E:T) cell ratios (˜1-5 CAR⁺ T cells per 100 tumor cells), CAR-T cells generated from SINV-CAR/tFab eliminated far more (up to ˜6-fold) tumor cells within 4 days than CAR-T cells generated from SINV-CAR alone (FIGS. 9C & 13D). A similar trend was observed using another CD19⁺ tumor cell line (Daudi). The observed cytotoxic effect was consistent with the detection of IFN-γ and IL-2 in the culture medium collected within 24-48 hours of co-culturing (FIGS. 9D & 13E).

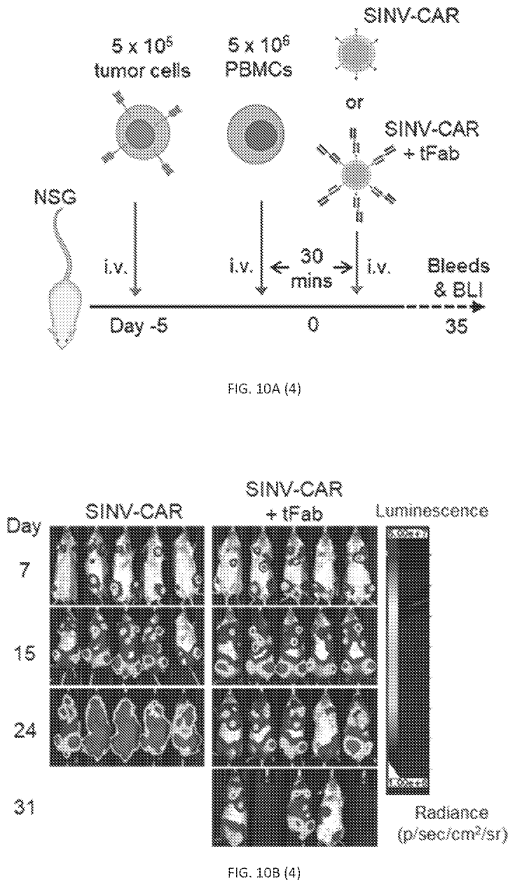

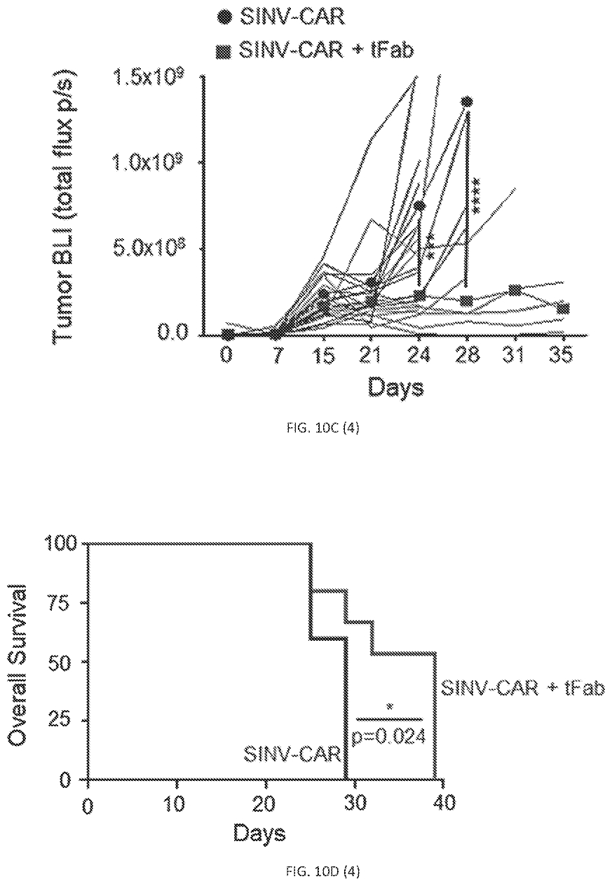

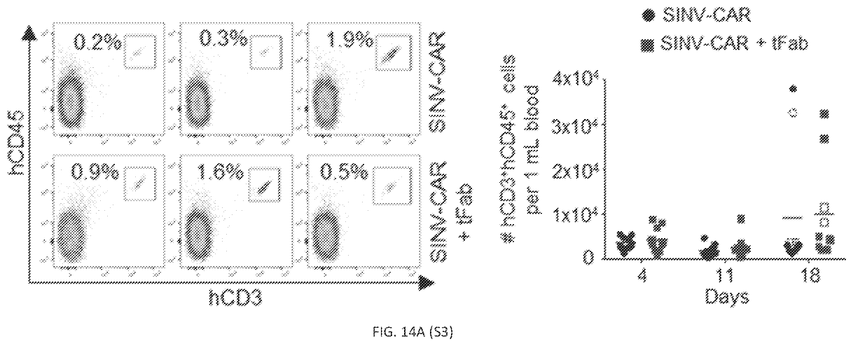

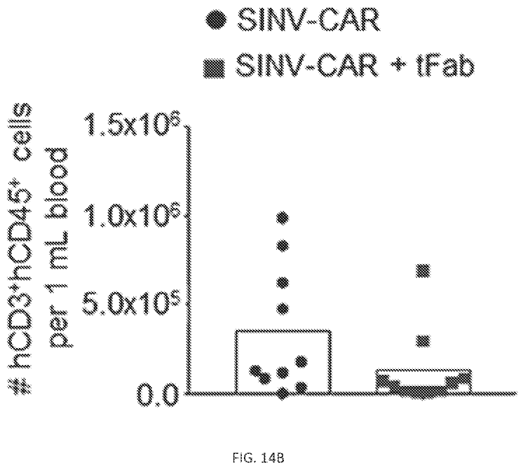

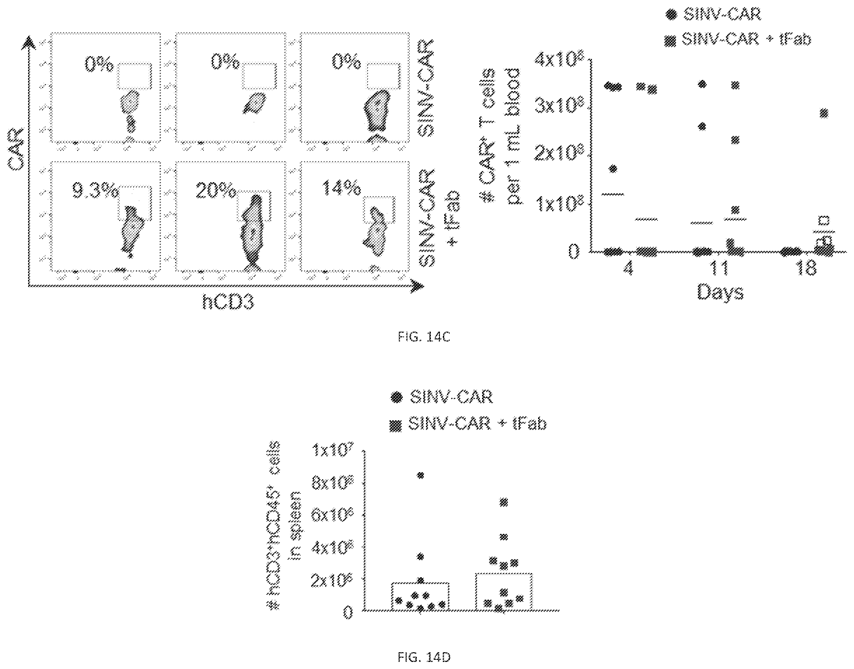

The efficacy of the SINV-CAR/tFab vector system was evaluated in a xenograft mouse model (FIG. 10A). CD19⁺ BV-173 cells, engineered to express firefly luciferase as imaging reporter to allow monitoring of tumor growth in vivo, were engrafted into NSG mice. Five days later, activated human PBMCs were injected intravenously into the animals, followed by SINV-CAR with or without tFab 30 minutes later. By day 24 following SINV-CAR injection, mice treated with SINV-CAR/tFab displayed significantly reduced tumor bioluminescence (BLI) compared to control mice infused with SINV-CAR alone (FIGS. 10B & 10C). Control mice began developing hind-limb paralysis due to tumor localization in the spine, which necessitated sacrificing all animals at a much earlier time point (10 days earlier according to median survival times) than mice treated with SINV-CAR/tFab (FIG. 10D). An attempt to quantify CAR⁺ and CD3⁺ human T cells circulating in the peripheral blood was made. While only very small numbers of CAR⁺ and CD3⁺ human T cells were detected at early time points (FIGS. 14A & 14C), substantial quantity of CAR⁺CD3⁺ human T cells was found in the peripheral blood of all mice treated with SINV-CAR/tFab at the time of sacrifice (FIG. 10E). These greater levels of CAR⁺CD3⁺ human T cells were attributed to greater T cell transduction by SINV-CAR/tFab vs. SINV-CAR and not attributed to simply greater total number of T cells in the peripheral blood, as total T cell counts were similar between both treatment groups (FIG. 14B).

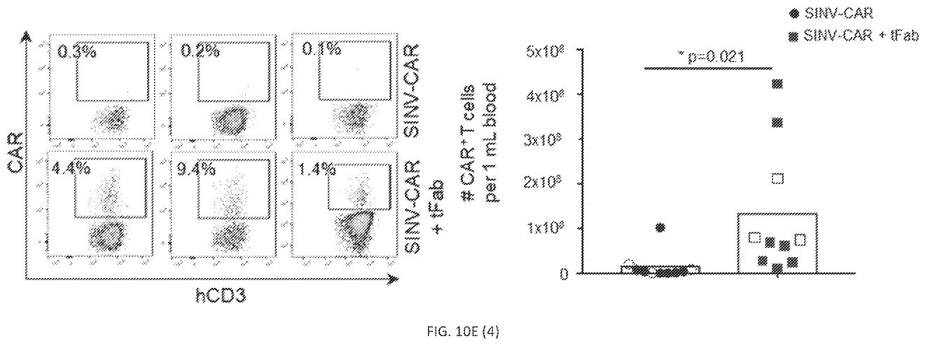

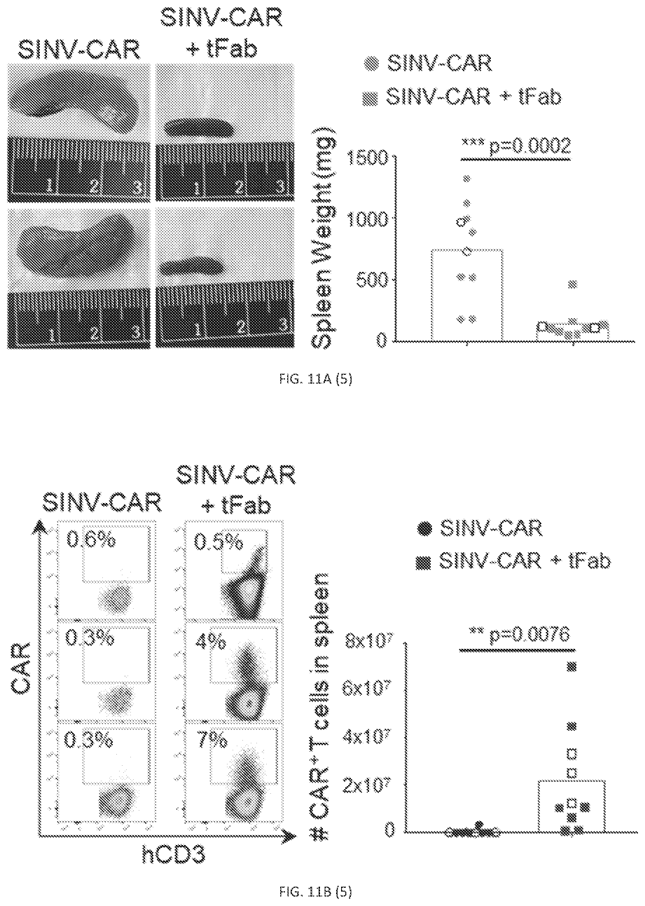

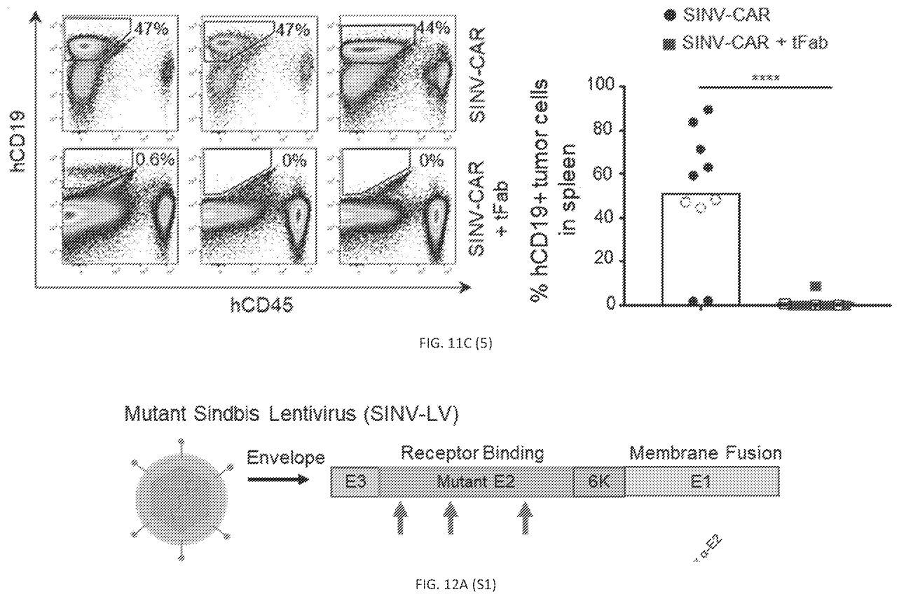

The aggressive BV-173 B cell lymphoma model appears to result in accumulation and spread of tumor cells in the spleen: at the time of sacrifice, very enlarged spleens were discovered in mice treated with SINV-CAR alone (FIG. 11A), with a very high proportion of BV-173 tumor cells in the enlarged spleens (>50% of the total cell populations on average) (FIG. 11C). In contrast, the overall size and weight of spleens from mice treated with SINV-CAR/tFab appeared comparable to those from normal, healthy mice. Analysis of the cellular composition of the spleens revealed higher infiltration of CAR-T cells in mice treated with SINV-CAR/tFab (FIGS. 11B & 14D), which correlated with much lower numbers of CD19⁺ BV-173 tumor cells (<1% on average) (FIG. 11C). Taken together, these data suggest that generating even a relatively small number of CAR-T cells directly in vivo is sufficient to enable tumor suppression in lymphoid organs and significantly prolong the median survival time of tumor-bearing mice.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A gene delivery system comprising
a viral gene delivery vector particle comprising a polynucleotide encoding at least one gene-of-interest; and
at least one bispecific polypeptide configured to bind a viral gene delivery vector particle and a target cell-specific receptor protein,
wherein the viral gene delivery vector particle is a lentivirus.

2. The gene delivery system of claim 1, wherein the lentivirus comprises a modified Sindbis virus envelope protein unable to bind a cell surface protein.

3. The gene delivery system of claim 1 or 2, wherein the bispecific polypeptide comprises at least one binding domain configured to bind the viral gene delivery vector particle and at least one binding domain configured to bind the target cell-specific receptor protein.

4. The gene delivery system of claim 3, wherein the bispecific polypeptide further comprises a flexible linker covalently joining the two binding domains.

5. The gene delivery system of any of claims 1-4, wherein the bispecific polypeptide is an antibody, or fragment or derivative thereof.

6. The gene delivery system of claim 5, wherein the antibody comprises a human or humanized antibody.

7. The gene delivery system of any of claims 1-6, wherein the bispecific polypeptide comprises two Fab domains individually configured to bind the viral gene delivery vector particle and the target cell-specific receptor protein.

8. The gene delivery system of any of claims 1-7, wherein the bispecific polypeptide binds the modified Sindbis virus envelope protein.

9. The gene delivery system of claim 8, wherein the bispecific polypeptide binds the modified Sindbis virus envelope protein E2 domain.

10. The gene delivery system of any of claims 1-9, wherein the target cell-specific receptor protein is selected from the group consisting of a T cell receptor, a B cell receptor, and a cancer cell marker.

11. The gene delivery system of claim 10, wherein the T cell receptor comprises CD3, CD4, or CD8.

12. The gene delivery system of claim 10, wherein the B cell receptor comprises CD19.

13. The gene delivery system of claim 10, wherein the cancer cell marker comprises HER2.

14. The gene delivery system of any of claims 1-13, wherein the at least one gene-of-interest comprises a marker protein, a therapeutic protein, elements required for genomic editing or gene silencing, or a combination thereof.

15. The gene delivery system of any of claims 1-14, wherein the at least one gene-of-interest comprises a chimeric antigen receptor.

16. A composition comprising
a viral gene delivery vector particle comprising a polynucleotide encoding at least one gene-of-interest; and
at least one bispecific polypeptide configured to bind a viral gene delivery vector particle and a target cell-specific receptor protein,
wherein the viral gene delivery vector particle is a lentivirus.

17. The gene delivery system of claim 16, wherein the lentivirus comprises a modified Sindbis virus envelope protein unable to bind a cell surface protein.

18. The gene delivery system of claim 16 or 17, wherein the bispecific polypeptide comprises at least one binding domain configured to bind the viral gene delivery vector particle and at least one binding domain configured to bind the target cell-specific receptor protein.

19. The composition of any of claims claim 16-18, wherein the bispecific polypeptide further comprises a flexible linker covalently joining the two binding domains.

20. The composition of any of claims 16-19, wherein the bispecific polypeptide is an antibody, or fragment or derivative thereof.

21. The composition of claim 20, wherein the antibody comprises a human or humanized antibody.

22. The composition of any of claims 16-21, wherein the bispecific polypeptide comprises two Fab domains individually configured to bind the viral gene delivery vector particle and the target cell-specific receptor protein.

23. The composition of any of claims 16-22, wherein the bispecific polypeptide binds the modified Sindbis virus envelope protein.

24. The composition of claim 23, wherein the bispecific polypeptide binds the modified Sindbis virus envelope protein E2 domain.

25. The composition of any of claims 16-24, wherein the target cell-specific receptor protein is selected from the group consisting of a T cell receptor, a B cell receptor, and a cancer cell marker.

26. The composition of claim 25, wherein the T cell receptor comprises CD3, CD4, or CD8.

27. The composition of claim 25, wherein the B cell receptor comprises CD19.

28. The composition of claim 25, wherein the cancer cell marker comprises HER2.

29. The composition of any of claims 16-28, wherein the at least one gene-of-interest comprises a marker protein, a therapeutic protein, elements required for genomic editing or gene silencing, or a combination thereof.

30. The composition of any of claims 16-29, wherein the at least one gene-of-interest comprises a chimeric antigen receptor.

31. A method of transducing a cell with at least one gene-of-interest, comprising contacting a cell expressing the target cell-specific receptor protein with the gene delivery system of any of claims 1-15 or the composition of any of claims 16-30.

32. A method of targeting at least one gene-of-interest to a cell or tissue, comprising administering to a subject having a cell or tissue expressing the target cell-specific receptor protein the gene delivery system of any of claims 1-15 or the composition of any of claims 16-30.

33. The method of claim 31 or claim 32, wherein the at least one gene-of-interest comprises a chimeric antigen receptor.

34. A method of generating CAR-T cells in vivo, comprising administering to a subject the gene delivery system of any of claims 1-15 or the composition of any of claims 16-30,
wherein the at least one gene of interest comprises a chimeric antigen receptor and the target cell-specific receptor protein is a T cell receptor.

35. The method of claim 34, wherein the T cell receptor is selected from the group consisting of CD3, CD4, CD8, or a combination thereof.

36. A method of treating a disease or disorder, comprising administering to a subject in need thereof an effective amount of the gene delivery system of any of claims 1-15 or the composition of any of claims 16-30,
wherein the at least one gene of interest comprises a chimeric antigen receptor, a therapeutic protein, or a combination thereof.

37. The method of claim 36, wherein the disease or disorder comprises cancer.
